(19) United States
(12) Patent Application Publication
(10) Pub. No.: US 2003/0032029 A1
(43) Pub. Date: Feb. 13, 2003

(54) Three dimensional apparatus and method for integrating sample preparation and multiplex assays

(75) Inventor: Mark L. Collins, San Diego, CA (US)

Correspondence Address: LYON & LYON LLP, 633 WEST FIFTH STREET, SUITE 4700, LOS ANGELES, CA, 90071, US

(73) Assignee: Nanogen, Inc., San Diego, CA (US)

(21) Appl. No.: 10/096,718

(22) Filed: Mar. 12, 2002

Related U.S. Application Data
(63) Continuation of application No. 09/217,472, filed on Dec. 21, 1998, now abandoned.

(51) Int. Cl. C12Q 1/70; C12Q 1/68; C12M 1/34

(52) U.S. Cl. 435/6; 435/5; 435/287.2

(57) ABSTRACT

A method and apparatus are provided for integrating sample preparation and multiplex assay of high volume samples for the presence of nucleic acid and antigen targets. The method and apparatus use a three dimensional platform, such as a column, for capturing desired targets out of the large volume sample. The column has a multiplicity of sample processing and target capture zones. The method and apparatus further provides a simple and efficient sample pre-processing and testing methodology, as well as a simple and environmentally friendly detection methodology.

### FIELD OF THE INVENTION

This invention relates generally to multiplex systems for carrying out fully integrated clinical diagnostics, combining sample preparation, nucleic acid hybridization reactions and antibody/antigen reactions. More specifically, this invention relates to systems utilizing column technology wherein a column having discrete sample preparation zones, and capture/detection zones for nucleic acid and/or antigens is designed for multiplex high volume assays of clinical samples.

### BACKGROUND OF THE INVENTION

The development of sensitive and easy to use detection methods has been an ongoing goal of practitioners in the clinical diagnostic arts. Towards this end, advances in the molecular biology arts have provided the basis for many highly sensitive detection methods for single nucleic acid analytes such as HWV RNA, including direct detection methods such as the branched DNA technology (Collins et al., Nucleic Acids Research 25, 2979-84,1997). This method can detect and quantify target nucleic acids down to 50 copies/mL (100 zeptomolar). This method does not require target amplification, is highly sensitive and quantitative, but is unsuitable for the present invention because it requires a centrifugation step prior to assay, an overnight incubation, and is not suitable for simultaneous multiplex analysis of nucleic acids and antigens.

Recent advances in the molecular biological arts have further provided practitioners the ability to perform multiplex detection of thousands of targets in extremely compact systems, such as the high density arrays disclosed by Fodor et al. in U.S. Pat. No. 5,744,305. However, such an array is not suitable for the present invention because its sensitivity is poor (in the high femtomolar to low picomolar range) and it requires a complex sample preparation procedure as well as target amplification. Moreover, the method is not suitable for simultaneous assaying of nucleic acids and antigens.

Notwithstanding advances in technology as mentioned above, there has been no advancement that has proven applicable to multiplex detection for both high volume samples as well as nucleic acids, proteins, and haptens. For example, in analyzing patient blood samples for HIV viral load, it is also desired that such sample be tested for viral drug resistance, drug monitoring, and for the health of the immune system. This requires both extreme sensitivity (<500 copies viral RNA/mL) as well as simultaneous quantification of three different analytes (viral RNA, CD4+antigen, and drug metabolites). Existing art requires that assays for such different diagnostics be done separately, often in different laboratories. The information obtained has to further be compiled by the physician and the treatment strategy adjusted accordingly. The current invention addresses the desire that such testing be carried out using a single sample preparation and assay method to collect all of the information from a whole blood sample in one procedure thereby improving efficiency and minimizing the chances for errors, confusion, or misinterpretation of results. Moreover, a single report can, for example, highlight potential contradictions and indicate the need for re-testing, something a physician could miss while digesting diagnostic information from three separate reports. The drug monitoring section of the report can highlight failure of the patient to take the drugs, or detect underdose or overdose of the drug, or additionally disclose an adverse side reaction to a drug.

Another problem with currently available methods has been the time to first result. In cases of blood poisoning, for example, it is desirable to obtain detection rapidly (e.g., less than one hour). With existing technology, rapid detection has been elusive because of either the need to culture suspect organisms or at the very least the need to carry out time-consuming sample preparation steps and amplification of target molecules. Thus, there is a need in the art for a rapid, ultra-sensitive direct assay with a simple and rapid sample preparation procedure.

The above stated goals have not been met by existing art in yet another aspect. With respect to nucleic acid assays, classical hybridization detection methods have relied on passive hybridization, which is both slow and inefficient. For example, typical multiplex hybridization formats involve passive hybridization in one dimension, i.e., capture molecules are anchored to a microspot (a “point”) on a two dimensional planar platform, while the target molecules are in a three dimensional solution. Such a situation causes the actual hybridization to occur at the one dimensional level i.e., hybridization at a single point. Additionally, by the nature of their extremely small sized arrays, chip systems cannot handle volumes of greater than a few microliters, generally 5 to 25 μL. Such systems are impractical for use in detecting targets in samples that are one mL or greater in volume unless target amplification procedures are performed. Concentration of most samples from one mL to approximately 10 μL is not practical because the resulting concentration of nucleic acid and protein is so high that the rates of reaction are reduced and the background is greatly increased.

Chip technology is also not well suited to carrying out simultaneous assays on nucleic acids and antigens. Currently, nucleic acid probe assays and the polypeptide immunoassays are either done sequentially on the same chip or on different chips. Such requirements are time consuming because samples must be split (which can also reduce sensitivity) and prepared for the different assays separately prior to detection.

Other assay systems have also been developed with similar shortcomings. For example, Kenney et al., Biotechniques 25:516-521, 1998, disclose a method (i.e., the acrydite method) wherein labeled targets from samples are subjected to electrophoresis through a gel having capture probes that are immobilized within discrete zones. Upon encountering the complementary probe, the target is captured and detected in the gel. Such a system is not applicable to high volume samples or samples that have a high concentration of non-target nucleic acid molecules. Moreover, such systems are not easily amenable to either signal amplification or to detection of protein targets, as in immunoassays, due to variability in the charge of individual protein target molecules that may need to be electrophoresed through the gel.

In another example, Müller and Lane, U.S. Pat. No. 5,804,384 disclose a device for performing multiplex assays consisting of a capillary tube filled with a linear array of discrete points for capturing target molecules separated by optional inert material. As the sample is re-circulated through the capillary, the target is captured by hybridization to target-specific capture probes along the capillary. The target is labeled by PCR prior to injection or by hybridization to an amplifiable reporter probe such as midivariant RNA (using Qbeta replicase). The preferred method of detection is fluorescence. This system is unsuitable for the present invention because a separate device must be made for each panel; it cannot handle milliliters of sample potentially containing particulates; the sample preparation is not integrated into the device; and it is difficult to perform both immunoassay and probe assays on the device since the sample preparation, the temperature, and salt requirements are very different.

In light of the above referenced methodologies, it is desired to overcome the inabilities of existing art so as to combine sample preparation and multiplex detection of target species from large volume samples (i.e., greater than one ml). We have discovered such a method and apparatus to perform sensitive detection of target molecules derived from high volume environments using a simple multiplex assay in a column format.

The column approach has many advantages over both the two dimensional chip and linear capillary arrays (i.e., one dimensional or point source capture). The first advantage is that samples are to be prepared and assayed in a single buffer solution, allowing simplification of sample testing. Sample preparation is integrated into the device. The second advantage is that large sample volumes are to be handled readily without loss of sensitivity or a need to reduce the complexity of the sample solution (i.e., removal of non-target species), or a need to concentrate the sample. A third advantage is that the current invention need not require amplification of nucleic acid target species.

A fourth advantage is that manufacturing requirements are simplified because the target molecule capture methodology involves the use of individual capture zones which are applicable generically to many test panels for a multiplicity of target species. For example, each zone has its own capture probe sequence and every column may be manufactured so as to have the same number of capture zones. Some assays may use all of the zones, while other assays use only a few of the zones. One result of such an array of zones is that the time to perform an assay is the same whether there are only a few targets to be detected or many.

In yet a fifth important advantage, the column format allows the entire sample volume to be forcibly exposed to all of the target binding sites rather than a tiny fraction of them. The ability to re-circulate can be used to improve the efficiency of both target capture and labeling. This in turn allows the size of the capture zones to be reduced, which improves sensitivity by reducing backgrounds and increasing signal intensity.

In a sixth important advantage, the capture nucleic acid and antigen targets are exposed on the surface of the three dimensional column packing material for efficient signal amplification. The packing material is referred to generally herein as “three dimensional platforms” or alternatively “three dimensional capture probe platforms”. By “three dimensional” is meant the common dictionary term wherein an object fills a three dimensional space. By “capture probe platforms” or simply “platforms” is meant a space filling material such as a solid support upon the surface of which is contacted capture probes which may be generally, nucleic acids and/or polypeptides. As described in more detail below, the capture probe platforms may further comprise three dimensional arrays of capture probes contacting or otherwise associated with the platforms or solid supports.

In a seventh important advantage, the column method not only allows for a single sample preparation method, but also for facile removal of both sample-derived insoluble matter and particulates and other undesirable reagents originating from the sample preparation buffer (e.g., bead-immobilized enzymes, salts, detergents, as well as substances that interfere with assays that require a single continuous flow process).

In an eighth important advantage, the present invention increases the confidence in results because they can be double-checked for correctness and consistency by using the combination of multiplex nucleic acid probe assays and multiplex polypeptide and hapten immunoassays.

In a ninth important advantage, the present invention makes it easier to complete panels and still maintain a simple sample preparation protocol because for each analyte there are two methods to detect its presence. For example, a respiratory disease panel is difficult to complete with nucleic acid probe assay technology alone because target pathogens such as Mycobacterium tuberculosis (Mtb) may be extremely difficult to lyse efficiently, making the sample preparation protocols very laborious. However, in the present invention, an ultrasensitive immunoassay may be incorporated to assay hard to disrupt macromolecular structures, such as the Mtb cell wall, in conjunction with testing for target molecules with nucleic acid probes thereby completing a spectrum for testing at both the genetic and protein levels.

In a tenth important advantage, a thorough digestion of the sample prior to immunoassay allows the selection of abundant internal viral and internal cellular antigens as well as the more typical coat protein and extracellular antigens. In addition to being of higher titer, the internal antigens are often better conserved than the external antigens since they are not under selective pressure to evade immune defenses.

### SUMMARY OF THE INVENTION

The present invention relates to a three dimensional method, and apparatus that can actively carry out sample preparation and controlled multiplex detection of target molecules from high volume samples in a column format. The method contemplates, among other elements, use of nucleic acid hybridizations and antibody/antigen reactions.

In a preferred embodiment, the invention uses three dimensional arrays of capture probes associated with solid supports. In one such embodiment solid supports carrying capture probes are themselves three dimensional (e.g., a spherical bead). The solid supports carrying the capture probe molecules are further arranged in multiples of spaced layers (i.e., capture layers/zones). In such arrangement, each such layer or zone has capture probes that are unique to that layer.

In another preferred embodiment, each spaced capture zone is separated by an inert opaque spacer layer. Such opaque spacer layer may comprise any inert material such as opaque colored beads or a filter that will allow target molecules, probes and labels to pass through the layer. In a preferred embodiment, the inert layer physically separates one capture zone from another. In another preferred embodiment, the opaque quality of the inert layer reduces “cross-talk” between the capture zones arising from light-generating reactions to detect the presence of target molecules within a capture zone.

In a preferred embodiment, a single apparatus possesses a plurality of capture zones for capturing nucleic acid targets or for capturing antigens such as polypeptide targets.

In another preferred embodiment, with respect to nucleic acid capture probes of each capture zone, such probes are engineered for specificity for a nucleic acid capture mediator probe (cmp). In such embodiment, the cmp is designed to have specificity for both the capture probe of a capture zone and for a nucleic acid target molecule. In a related preferred embodiment, the nucleic acid capture probes of a capture zone intended for protein targets are engineered for specificity for a protein capture mediator probe (pcmp) (e.g. an antibody) by labeling the pcmp with a nucleic acid that is complementary to the capture probe of such capture zone.

In another preferred embodiment, the capture probes are bound either covalently or noncovalently to the three dimensional supports of the capture zones. Covalent linkage can be carried out by any of a number of standard methods, including the reaction between an amine and either an aldehyde, an NHS ester, an isothiocyanate, or an activated COOH group. Another useful reaction is that of a thiol and either a maleimide or a haloacetamide (Pierce Chemical Company Catalogue, Chapter on Cross-linking reagents). Noncovalent linkage may be carried out by binding biotinylated probes to steptavidin-derivatized surfaces or by binding antigen-labeled DNA molecules to immobilized antibodies. In any case, the chemistry of attaching probes to solid supports is well known in the art.

In another preferred embodiment, the solid supports within the capture zones are transparent to visible light. This can be accomplished by using any number of different solid supports including glass, acrylic and polystyrene beads.

In another preferred embodiment, the invention contemplates use of a single buffer system for analysis of both nucleic acid targets and polypeptide targets. In a preferred embodiment, the buffer is a low salt or low ionic strength buffer. Preferably, a low salt buffer is optimal in using chimeric molecules, such as peptide nucleic acid molecules (“PNA”), as hybridization probes. PNAs may be used as cmps as well as probes used in signal amplification (label mediator probes lmp and labeled probes lp (see FIG. 1). PNAs may also be components of chimeric PNA-DNA preamplifier and amplifier probes.

In another embodiment, the apparatus of the invention has a multiplicity of pre-capture zones for (1) filtering out particulates in the crude sample mixture or sample preparation reagents, (2) performing enzymatic digests of the sample, and (3) removing denaturants and interfering substances. Another embodiment of the invention provides for ports for re-circulating or exporting sample solutions that have passed at least once through the apparatus.

In yet another embodiment the apparatus of the invention provides for zones to capture and remove from the sample solution cmps and lmps not bound to targets.

In still another embodiment, the apparatus of the invention provides for a means to heat sample solutions to a temperature of about 60 to 100° C.

In still another embodiment, the invention contemplates the use of pressure to assist the movement of the target molecules within a sample solution through the capture zones.

In a further embodiment, the invention contemplates use of a means to detect electromagnetic emissions emanating from the capture zones. Such a means may be any standard light detection device including a detector that can read a continuous emission over a period of time or one that can detect brief “flashes” of light energy. Application of such detectors is routine and well known in the art. Also contemplated is the use of cylindrical detectors for efficient 360° collection of the light.

### BRIEF DESCRIPTION OF THE DRAWINGS

FIG. 1 is a schematic showing five different formats for capturing and labeling nucleic acid targets.

FIG. 2 is a schematic showing a competitive immunoassay with three different levels of signal amplification for antigens of different titers.

FIG. 3 is a schematic diagram showing one embodiment of the column apparatus and various component parts thereof.

FIG. 4 is a schematic showing the column containing the spaced capture zones inserted into a cylindrical light emission detector.

FIG. 5 is a schematic showing another embodiment of the invention wherein the column device is designed as an annular column providing the invention with the capacity to use a light detection device that may be inserted into the center of the column in addition to an external detector as shown in FIG. 4.

FIG. 6 is a schematic showing capture of target using multiple capture mediators on a polymeric capture probe containing a repeated sequence.

FIG. 7 is a schematic showing one method of capturing a target to a solid support using cruciform capture mediator probes

FIG. 8 is a schematic showing a sandwich method of capture and labeling of an antigen

FIG. 9 is a schematic showing a signal amplification method for nucleic acid targets using cruciform label mediator probes, preamplifier, amplifiers, and label probes.

### DETAILED DESCRIPTION OF THE INVENTION

An apparatus is provided for sample preparation and the multiplex detection of target molecules derived from a sample. By sample pre-preprocessing is meant the addition of the sample to the column or the addition of reagents to the sample by the user and the addition of this mixture to the column. By sample processing is meant the conversion of a raw sample to one that is properly prepared for immunoassay and nucleic acid probe assay. By multiplex detection is meant the detection in a single assay of any of a number of target molecules originating from any of a number of specified sources that may be present in a single sample. By target molecules is meant generally, nucleic acids and antigens. By nucleic acids is meant any polymer of nucleobases, including DNA, RNA, 2′ Omethyl RNA molecules, whether naturally or synthetically derived or a polymer of nucleobases that comprise unnatural molecular components. By antigens is meant polypeptides and proteolytic fragments of polypeptides, polysaccharides, haptens, molecules such as drug metabolites or cellular metabolites, and any molecular structure that will bind to an antibody. By antibody is meant naturally occurring as well as synthetic polypeptide antibodies as well as non-polypeptide antibodies such as nucleic acid antibodies (aptamers). By sources is generally meant biological sources such as an organism or byproducts of an organism including prokaryotic and eukaryotic organisms. By sample is meant any material of an amount sufficient for testing for the presence in such material of target molecules. Generally, samples comprise material containing biological matter such as eukaryotic or prokaryotic organisms or materials from such organisms or from environments where such organisms may be found. By detection is meant the determination of the presence in the sample of a specific target molecule or quantification thereof. As is understandable to one skilled in the art of detection of molecules in a sample, the presence of any specific molecule in a sample may further indicate the presence of specific organisms or other macromolecular components in or associated with the environment from which the sample was taken.

In a preferred embodiment, the invention is used in the multiplex testing of relatively large volume samples (one milliliter or larger) for panels of molecular targets commonly associated with disease states. For example, stool samples from a patient suffering from an unknown disorder may be tested for a panel of suspect agents such as pathogenic organisms, toxins, metabolites, blood, etc. In another example, the purity of water may be tested for a panel of agents that are known to be associated with disease. In another example, whole blood drawn from a patient may be tested for a panel of suspect agents and metabolites or cellular components associated with any of a variety of disease states. In yet another example, foods may be tested for panels of organisms suspected in outbreaks of food-borne illnesses.

Sample pre-processing may vary depending upon the nature of the sample. However, the sample buffer generally comprises a standard formula. Certain criteria apply to both nucleic acid and polypeptide antigen targets. For example, reagents must be added to inactivate nucleases (particularly exonucleases) rapidly while at the same time it is desirable to prevent complete hydrolysis of polypeptide antigen targets. To accomplish this goal, one embodiment of the invention contemplates that the sample buffer include solid support-mounted enzymes for inactivating and/or digesting nucleases. Such enzymes may include trypsin to rapidly inactivate nucleases while at the same time partially digesting polypeptide target molecules to expose and preserve antigenic moieties on the polypeptides. Support-mounted enzymes are also useful to disrupt certain structures such as virus capsids. Bead-immobilized enzymes such as lysozyme and lysostaphin may further be used in samples containing microorganisms to degrade bacterial cell walls. Optimally, the sample buffer will also contain small molecular weight protein denaturants such as SDS to inhibit nucleases and expose them for better digestion by proteases. The SDS can also be of use in freeing antigens from pre-existing immune complexes.

The use of bead-immobilized enzymes further facilitates removal of the enzymes from the sample mixture upon contacting the sample with the column. Trapping of the immobilized enzymes at the top of the column further allows other “secondary” enzymes, such as those used in signal amplification, to avoid exposure to degrading enzymes that would otherwise likely be present if such enzymes were in free solution. Moreover, antibodies bound to solid supports in capture zones will also not be exposed to such enzymes. Another additional advantage of using bead-immobilized enzymes is that in environments requiring denaturants (e.g., SDS), immobilized enzymes are much more stable. The immobilization also allows incompatible enzymes such as proteases and their substrate (e.g., other enzymes) to work together in the same buffer without appreciably degrading one another.

Low molecular weight denaturants and detergents are preferred because they may be removed from the sample solution by porous size exclusion type beads within either pre-capture zones or the opaque spacer zones of the column. In a preferred embodiment, the bead-immobilized enzymes, denaturants and detergents are added to the sample preparation buffer individually as needed.

In another preferred embodiment, all sample preparation enzymes are arranged on the column in an optimal order. For example, high concentrations of SDS may be added to the sample pre-preparation buffer to quickly denature enzymes. This may be followed by a crude filtration and a zone to remove SDS. Then lysozyme and lysostaphin and enzymes to degrade fungal cell walls may occupy the next zone of the column. This may be followed by a zone containing for example, trypsin. The last enzymatic zone may be DNAse and RNAse, since the RNA and DNA will have been fully exposed by the previous zones. Optimally, the sample would be re-circulated through the sample preparation zones to maximize the effectiveness of the digestions.

Typically, the sample pre-preparation buffer would contain probes for nucleic acid targets, labeled antigens for competitive immunoassays, a low ionic strength buffer such as 50 mM Tris-HCl, pH 7.5, 5 mM EDTA, SDS between 0.1% and 2%, and possibly beads with bound trypsin, and beads with various other bound enzymes that will depend on the sample type. Typically, three or four mL of buffer would be added to one mL of sample (or one gram of sample such as a tissue homogenate). This dilution facilitates dissolution of pre-existing complexes and enzymatic digestion, increases solubility of all components, and reduces sample viscosity for better flow through the column.

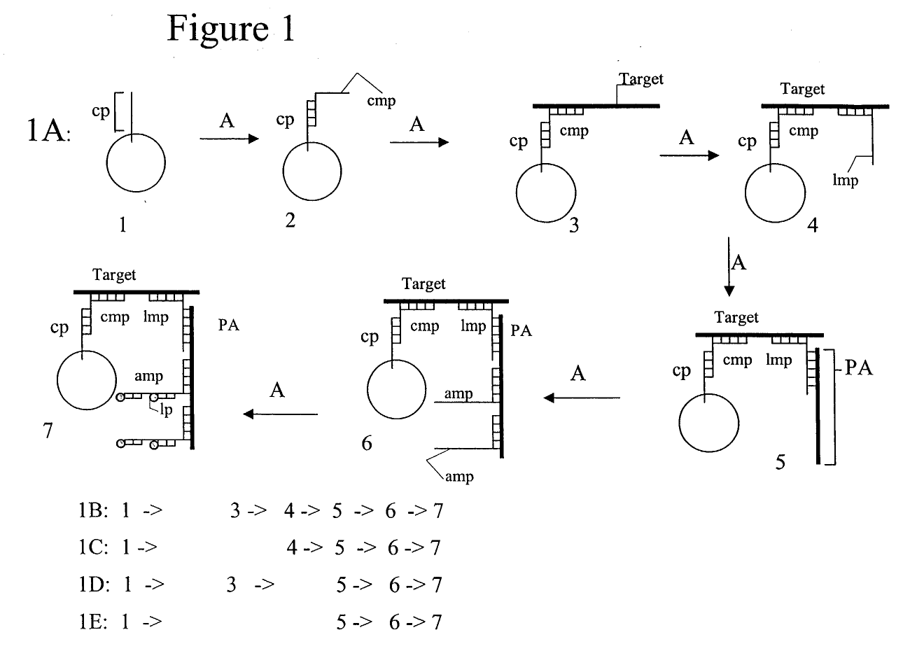

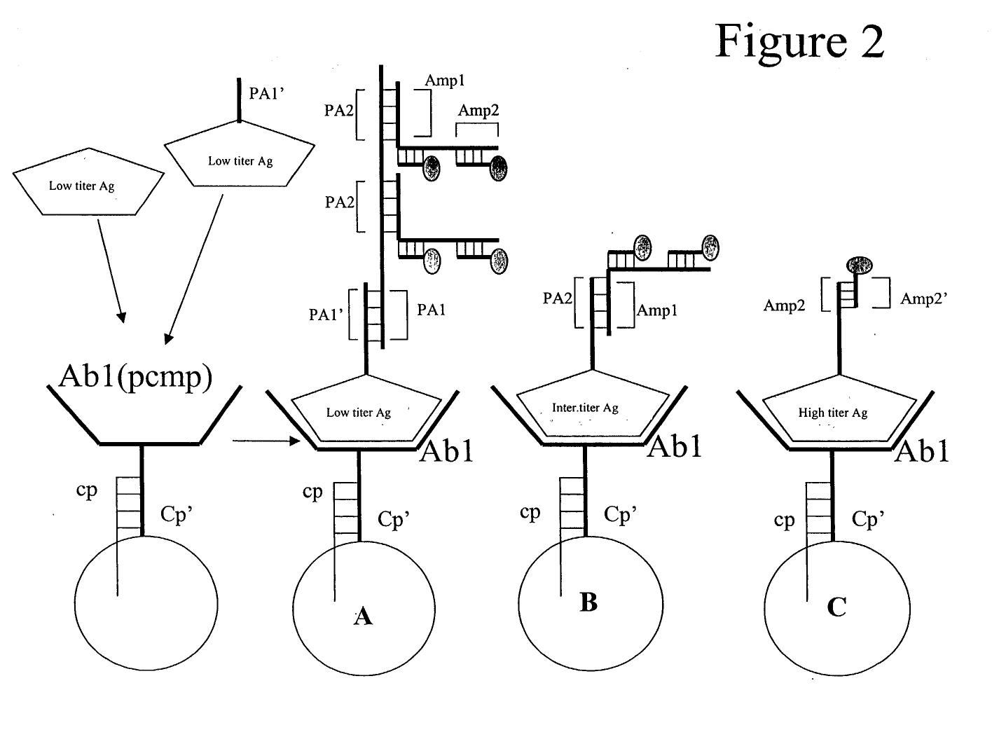

The capture mediator and signal amplification probes for nucleic acid targets are disclosed in FIG. 1 while those relevant to capture and signal amplification of targets for immunoassays are disclosed FIG. 2. FIG. 1A step 1 depicts a solid phase 1 containing a generic capture probe sequence cp. Each zone would have a unique cp sequence. In FIG. 1A step 2 a target-specific capture mediator probe cmp binds cp. In step 3 a target is captured by hybridization to cmp. In step 4 a label mediator probe lmp is hybridized to the target. In step 5 a preamplifier PA is hybridized to the lmp. In step 6 multiple amplifier probes amp are hybridized to the PA. In step 7 multiple labeled probes lp are hybridized to each amp.

Various steps defined in FIG. 1A can be combined. A number of preferred formats are indicated in FIGS. 1B through 1E. FIG. 1B indicates that the cmp can be hyridized directly to the target in the sample preparation solution so that step 2 of FIG. 1A may be omitted. FIG. 1C indicates that the cmp and lmp can be hybridized in solution to target so that steps 2 and 3 can be omitted and instead the target which is already complexed with cmp and lmp may hybridize as a single complex unit directly to the cp. FIG. 1D defines a format in which the cmp is hybridized to target in solution and the complex therefore captured directly on the solid support followed by addition of lmp and PA (omitting steps 2 and 4 of 1A). FIG. 1E defines a format in which cmp, lmp, and PA are hybridized to the target in solution to form a complex of target and probes prior to capture on the solid phase (omitting steps 2-4 of 1A).

FIG. 2 defines the preferred competitive immunoassay. In 2A, the protein capture probe pcmp antibody Ab1 is immobilized on the solid phase via cp and cp′ prior to assay. Cp′ is a nucleic acid sequence incorporated into the capture antibody. With regard to competitive immunoassay, low levels of an antigen labeled with a nucleic acid sequence PA′ complementary to PA is added which competes with unlabeled sample-derived antigen for binding the capture antibody Ab1 at a first epitope on the antigen. In subsequent steps the signal is amplified by hybridization of the PA, amp, and lp. In 2B an antigen of intermediate titer is labeled with a sequence complementary to the amp. This removes a layer of signal amplification. In 2C a high titer antigen is labeled with a sequence complementary to the lp. This removes two layers of signal amplification since the target antigen occurs physiologically at high titer.

In another embodiment, competitive immunoassay may not be desired and direct assaying for target carried out instead. In such case, the labeling of the antigen in 2A, 2B and 2C may be carried out by using an antibody specific for a second eptitope of the antigen. In this embodiment, the second antibody, rather than a competing antigen, is labeled with nucleic acid sequence that is complementary to either PA, amp, or lp.

In a preferred embodiment, the mediator probes (both cmp and lmp) are peptide nucleic acids (PNAs) with melting temperature (Tm) values in low salt of approximately 60-900° C. at 1 nM. The term PNA is used to refer to three chemically distinct types of peptide nucleic acid molecules, which are designated PNA1, PNA2, and PNA3. PNA1 is a PNA such as that referred to by Buchardt et al., in U.S. Pat. Nos. 5,786,461, 5,766,855, and 5,736,336, all of which are herein incorporated by reference, in which the sugar-phosphate backbone has been replaced by an uncharged, achiral modified peptide backbone. PNA2 is a sequence of negatively charged nucleic acid containing one or two pendant oligopeptide sequences to accelerate hybridization, such as the kind described by Corey, J. Am. Chem. Soc. 117:9373-4, 1995, herein incorporated by reference. A particularly preferred peptide sequence is (AAKK)₄, where A=alanine and K=lysine. For RNA targets a particularly preferred nucleic acid for PNA2 is 2′ Omethyl RNA, due to its higher Tm. PNA3 refers to a sequence of PNA also described in the above Buchardt et al. references, with one or two oligopeptide sequences described by Corey to accelerate hybridization. The use of any of the three types of PNA mediators facilitates hybridization in the low salt environment of the sample buffer solution mixture. The use of PNA further improves the quality of quantification results since the hybridization should be quantitative in low salt at high temperature.

Certain cationic, zwitterionic, and neutral polymers of nucleobases are also suitable for use as mediators in the present invention, provided they have a high Tm (>60° at 1 nM) in a low salt environment and hybridization specificity as good as DNA or better (Horn, T. et al., Tetrahedron Letters 37, 743-6,1996), herein incorporated by reference.

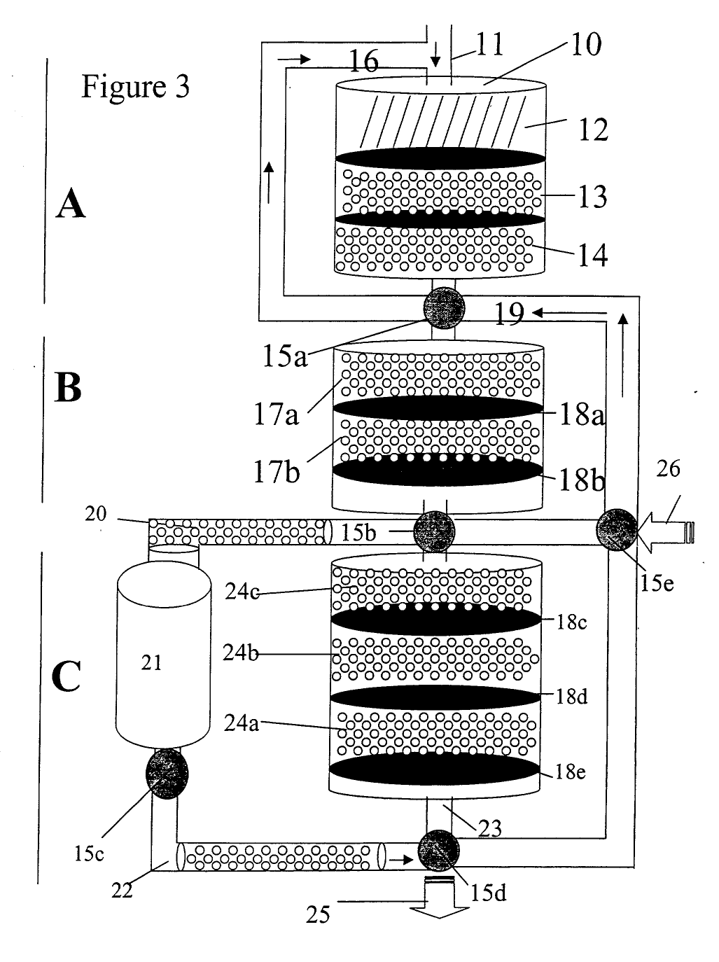

In FIG. 3 is provided a multiplex column device 10 having three sections A, B, and C. The sample is introduced through orifice 11. The upper section A comprises a plurality of sample preparation (pre-capture) zones that include, but are not limited to, a crude filtration zone 12, a zone 13 for removal of SDS and other substances that would interfere with assays, and at least one zone 14 for further sample digestion. Each of these zones performs a special function and the exact order of functional zones can be varied and some zones can be combined.

Zone 12 may comprise a nonporous matrix (e.g. glass wool) to trap particulates such as debris found in stool and food samples, or cellular material from digested tissue or disrupted cells such as microbes, blood, or tissue (e.g., biopsy). This zone is further useful for trapping various enzyme-derivatized beads that may be incorporated into the sample preparation protocol. The matrix making up zone 12 may also include certain immobilized enzymes that work optimally after proteolytic enzymes have been removed.

Zone 13 may comprise porous beads that bind any number of substances that are detrimental generally to immunoassays or nucleic acid probe assays. For example this zone would contain porous beads for capturing SDS which is detrimental to antibodies that must capture antigens. It is desirable not to capture all small ions at this time since some immunoanalytes are themselves small ions and the antibody assays generally perform better with salt present. (Removal of substances that interfere specifically with a given panel would be carried out by incorporating in the sample preparation buffer a means to remove such substances prior to loading the sample on the column).

Zone 14 optimally contains additional sample preparation beads, particularly ones that do not function as well in the initial sample preparation buffer solution. These might include beads with endonucleases (DNAse and RNAse) for partial digestion of target nucleic acids, which cannot work on the sample until proteases and detergents in the sample preparation buffer or in upstream zones of section A have had an opportunity to lyse or disrupt microbes or other cellular structures in the samples. DNAse in particular is very sensitive to high concentrations of SDS. These beads would optimally contain immobilized suitable buffer ions to provide the correct ionic strength and pH. Use of DNAses and RNAses provide a means whereby sample viscosity may be lowered to improve sample flow through the column as well as facilitating denaturation of RNA and DNA. The partial digestion of both types of nucleic acid further improves hybridization efficiency and target capture efficiency.

Following passage through zone 14, the sample passes through a valve 15a. The sample is optimally recycled through section A via 16 for further digestion prior to entering section B of the column in which immunoassays are performed. Section B comprises a multiplicity of capture zones. FIG. 3 depicts only two such zones for simplicity. Zones 17a and 17b may comprise a series of capture zones for capturing antigens by pcmp (e.g., antibodies). These zones may further comprise spacer zones 18a and 18b to physically separate the capture zones from one another so that cross-talk with respect to light-generating detection methods will be minimized. The spacer zones could be opaque (as pictured in the accompanying drawings) to prevent light from passing between capture zones or they could be highly reflective to enhance light output to the detector. Each spacer zone could in fact be multilaminar. They could be opaque in the center and highly reflective on the exterior of the spacer zones. For clarity FIG. 3 pictures thin spacer zones and wide capture zones. Preferably, the capture zones are thinner for greater sample concentration and more sensitive detection, and the spacer zones are wider than the capture zones to reduce cross-talk among the capture zones (to make the system more light-proof). In a further preferred embodiment, the capture zones are constructed as thin as efficient capture dynamics will allow. Efficient capture (>90% capture is desired) depends on efficient re-circulation and maximum acceleration of immuno-capture and hybrid-capture.

In a preferred embodiment, section B may have anywhere from 10 to 50 such capture zones. For example, additional zones may be included for measuring drug resistance and toxins by immunoassay.

Capture zones having pcmp (e.g., antibodies) may contain beads with immobilized buffer to maintain pH and ionic strength within the capture zones so as to provide an environment with sufficient osmotic pressure to keep the immobilized antibodies from denaturing in low salt environments. This is particularly important where signal amplification nucleic acids (e.g., preamplifier and amplifier) are to be used in amplifying the signal for section B as well as downstream section C.

The sample mixture may be directed out of section B through valve 15b and routed for re-circulation through the antigen capturing zones via port 19, or may be directed onto the next following sets of zones for nucleic acid capture in section C of the column. Optimally this section is physically separated from section B or thermally insulated so that nucleic acid section C may be kept at a higher temperature than antibody section B.

Following re-circulation, if desired, through section B, the sample mixture may be directed into a desalting zone 20 to prepare the sample mixture for nucleic acid heat-denaturation in zone 21. Ion exchange resin contained within beads with small pores is useful for irreversible deionization. Optimally these beads are preloaded with a buffer such as histidine to replace the salt with a zwitterion to maintain the pH near neutrality. Removal of ions from the sample will facilitate the thermal denaturation of nucleic acids for quantitative hybridization as well as increase the Tm of the preferred PNA probes to nucleic acid targets.

The fluid may be maintained in zone 21 by closing valves 15b and 15c. Heating element 21 is used to heat the sample to a temperature between 65 and 95° for denaturing nucleic acids. The sample may be allowed to incubate in element 21 at a temperature of between 60 and 800 to allow rapid and efficient hybridization of cmp and lmp (added to the sample prior to loading of the sample on the column) with the targets. Heating may be carried out for 30 seconds to 15 minutes. Optimally, the fluid entering section C of the column is at the temperature of the hybridization so that proper stringency can be maintained.

Following heat treatment, valve 15c is opened and zone 22 is optimally used to remove mediator probes not bound to targets. These unbound probes can cause background either by binding to solid support-bound capture probes or by nonspecifically binding to the solid supports of the capture zones. In one embodiment, the principle of size exclusion can be used to keep targets from entering the matrix of the beads used to eliminate unbound probes in zone 22. In another embodiment, the beads in zone 22 have bound thereto probes complementary to short universal sequences on the cmps and lmps that facilitate the removal of probes not bound to targets. Steric hindrance limits the capture of targets as disclosed in Collins, U.S. Pat. No. 5,780,224, herein incorporated by reference.

In one embodiment of the invention, chemical moieties are incorporated into the mediator probes that allow for the trapping of unbound probes within the size exclusion beads. For example, lysines on PNA probes could be trapped by ion exchange. Alternatively, oligohistidine (e.g. his₆ ) spacers on the probes could be trapped with Ni²⁺.

Following passage through 22, the fluid is directed into the column through entry port 23. The sample is then directed through a multiplicity of capture zones such as 24a-24c depicted in FIG. 3, each containing a unique capture sequence, and separated by spacer zones, 18c-18e. Optimally section C contains sufficient salt so that the negatively charged target can make a close and unhindered approach to the negatively charged capture probes on the solid phases. This facilitates rapid capture of target. Alternatively, sufficient salt can be added through port 26. Ideally the mediator probes have sufficient cations to reduce substantially the overall negative charge on the target fragments. The reduction in net negative charge of the target molecules was facilitated by fragmentation during sample preparation, since without this fragmentation the target would be much larger than the sum of the lengths of the cationic probes bound thereto.

Although in the example shown in FIG. 3, section C has only 3 capture zones, in a preferred embodiment, section C of the column may contain anywhere from 10 to 50 distinct capture zones. Different columns containing different numbers of capture zones could be prepared or one column containing the maximum number could be prepared and used for all assays. Generally, the number of capture zones needed in any one column will be based on the number of targets to be screened in any particular panel. For instance, a column intended for use in testing stool samples may comprise at least 7 capture zones wherein the zones are directed to capture selected nucleic acid target molecules derived from such organisms as Salmonella, Shigella, enterotoxigenic E. coli, Campylobacter, Yersinia, Listeria, and Vibrio cholerae.

As in section B, the spacer zones 18 in section C are used to physically separate the capture zones from one another so that cross-talk with respect to light-generating detection methods will be minimized. In a further embodiment, the spacer zones may comprise beads to facilitate additional removal of unbound mediator probes to further reduce background.

The sample may be re-circulated through the capture zones of the section C of the column via directing the flow of the sample through valves 15b, 15e and 15d, bypassing the external loop comprising elements 20, 21, and entrance port 22. Optimally, the sample may be re-circulated 3-10 times. This embodiment allows samples containing very low target molecule populations multiple opportunities to be captured by capture probes in the capture zones. Following capture, the non-binding portion of the sample mixture may be then directed out of the column and discarded through waste port 25.

In another embodiment, after washing sections B and C one or more times, signal amplification reagents not added earlier (e.g., lmp, PA, amplifier, label probes, and label substrate) may be added through entrance port 26. FIG. 3 pictures a single port 26 for reagent addition to sections B and C and a single waste port 25 for simplicity; sections B and C can have separate ports for addition and waste. By re-circulation through valves 15a, 15e, and 15b, Port 26 can be used to supply labeled antibodies for sandwich assays that do not need to pass through section C. Addition through the separate port 26 to sections B and C avoids contact of the reagents with proteases used in the sample preparation step that are trapped in the initial filter zone of section A.

In one such embodiment, signal amplification probes are added through port 26 in the following manner. The PA is directed through sections B and C and re-circulated if needed. Next, a wash solution is directed through sections B and C, followed by amplifier probe, followed in turn by another wash. Next, the labeled probe is added followed by substrate. The amplification reagents can be added through a single port and re-circulated separately through sections B and C at different temperatures (37° for section B and 50-60° for section C) or re-circulated through both sections B and C at the same temperature. After antibodies have bound their antigen, the thermal stability of the complex is much higher than that of the free antibody. It is thus possible to incubate section B at the high temperature optimum of section C (50-60°).

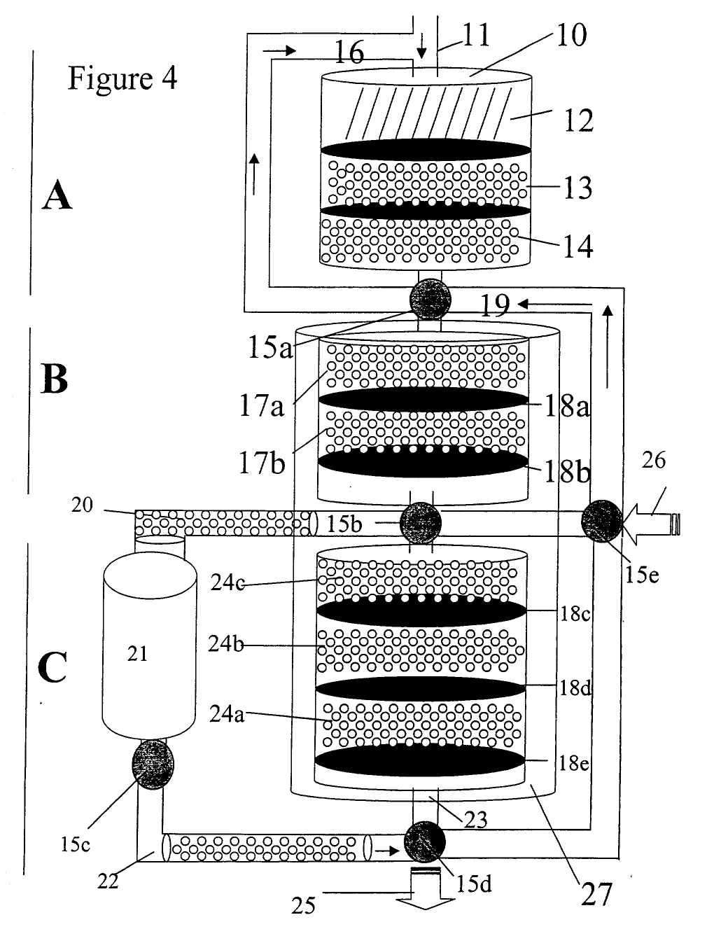

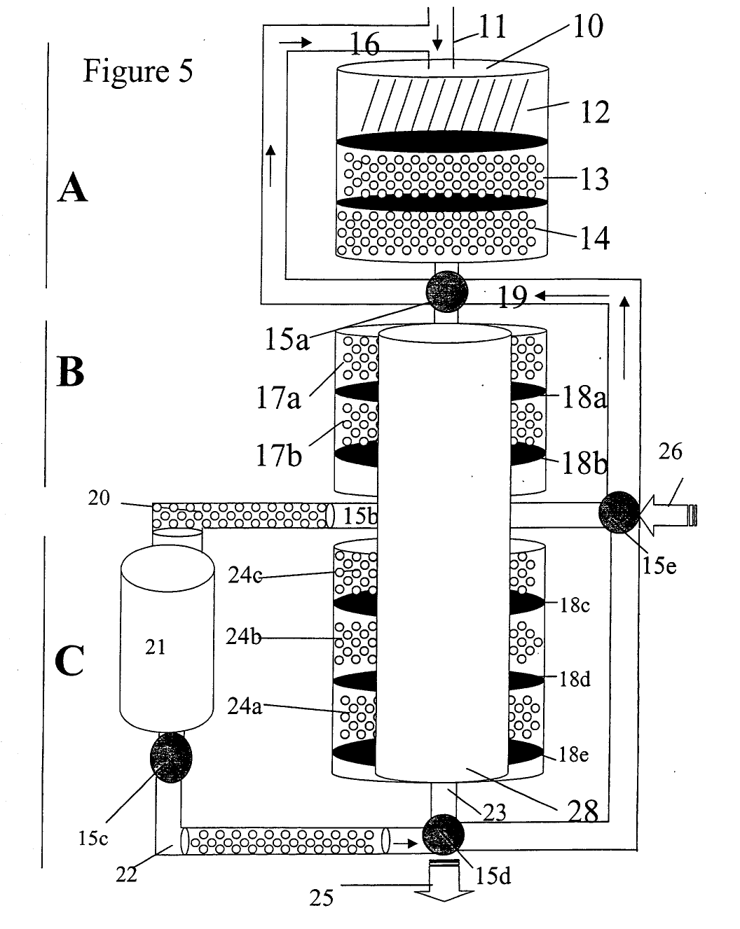

Optimally detection is done continuously for 1-10 minutes or alternatively it is done after a brief incubation. In FIG. 4 a cylindrical detector 27 for 360° collection of light is used for maximum sensitivity. In FIG. 5 an annular design of the column wherein the inner section 28 of the column is hollow may be used in conjunction with a light detector that may be inserted into the hollow space to increase the efficiency of light collection from the capture zones due to the reduction in the average distance from the light emitting source to the detectors. This design also facilitates the use of coincidence counting to control backgrounds in multi-photon signal generation schemes.

In an alternative embodiment, instead of a solid support comprising beads, the capture zones may use a solid support that is in the form of a porous filter. Additionally, the spacer zones may also comprise porous filter material. Use of this alternative facilitates the easy manufacture in stacking filters as desired.

The capture zones 17 and 24 of sections B and C respectively, are designed so as to have capture probes immobilized on solid supports therein. In a preferred embodiment, the capture probes are of a generally generic nature in that the capture probe of any one zone may be designed to have a sequence complementary not to specific target molecules but to mediator probes. Such a design will allow the number of capture probes that must be manufactured to be kept at a minimum. Such design further allows generic columns to be manufactured that will be applicable for use in detecting numerous target panels. Specifically, for example, a first capture zone will have a capture probe sequence designed to be complementary to a portion of a first cmp or pcmp sequence. A second capture zone will have capture probe sequence designed to be complementary to a portion of a second cmp or pcmp. Thus, a column designed to have for example ten capture zones will require the manufacture of only ten capture probes each designed to have a nucleic acid sequence that is complementary to a portion of either a different cmp or pcmp. Versatility in the column design is derived therefore from the ability for identical columns to be used for entirely different screening panels. Versatility is further derived from the design of the cmps and pcmps. Specifically, in addition to the portion of the cmp or pcmp that is complementary to the capture probe, a second portion of the cmp or pcmp is designed to be specific for an individual target molecule. Thus, only “reagent” materials (e.g., cmp and pcmp) need be manufactured in volume and variety.

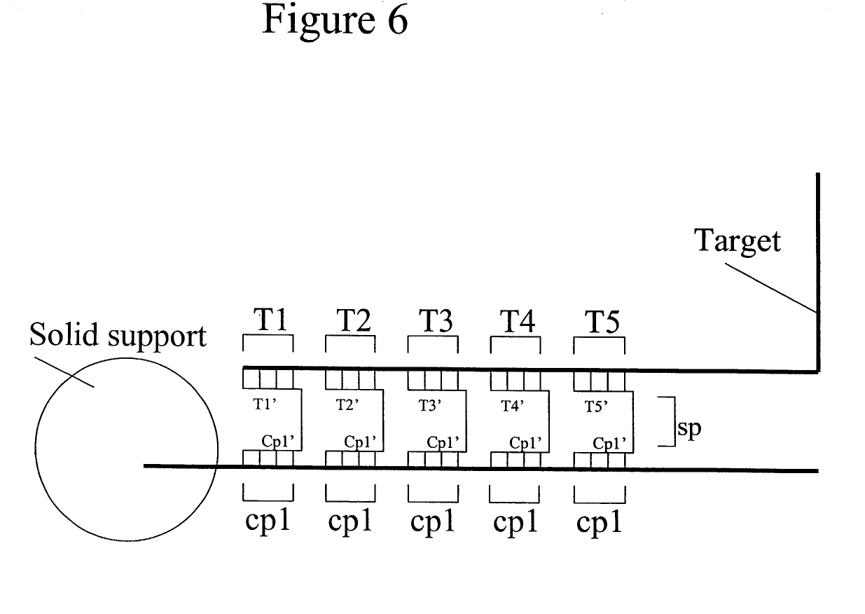

The design of the capture probes may take any number of forms. FIG. 6 shows one example wherein the capture probe comprises a directly repeating polymeric capture sequence (cp1) for capturing multiple cmps, each possessing one sequence (T1′, T2′, T3′, T4′, and T5′) complementary to a unique target sequence (T1, T2, T3, T4, T5 ) and another, (cp1′), that is complementary to the generic cp1 sequence. The advantage of this design is reduced steric hindrance in the capture of target molecules. Generally, nucleic acid cmps become annealed to the target prior to the sample passing through section C via incubation in element 21, and the generic sequence (cp1′) of the emps may be captured on the capture probe more easily which helps to accelerate the capture process (capture is initiated by capture through any one of the many capture mediators).

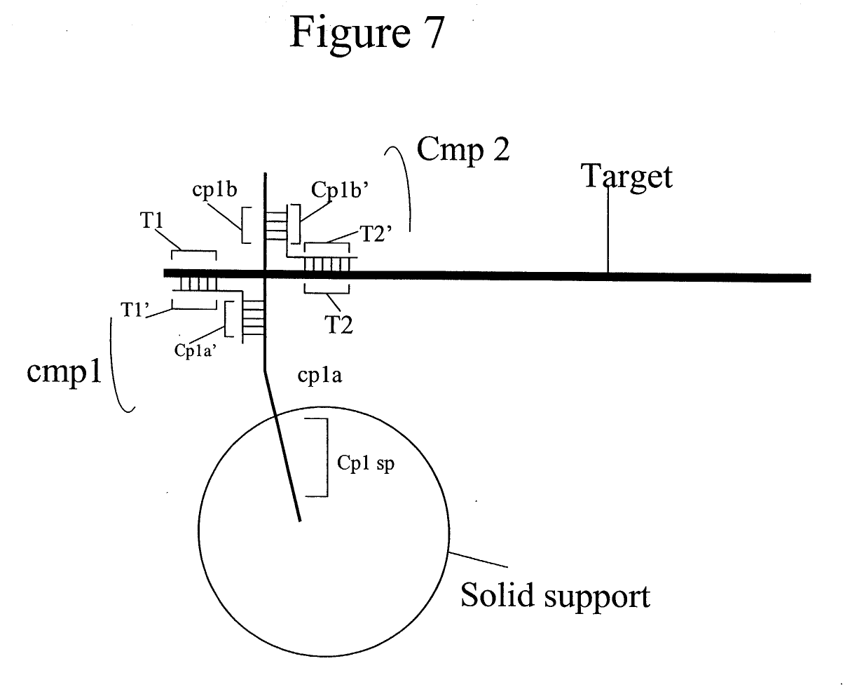

In an alternate design, pictured in FIG. 7, pairs of cmps (cm1 and cm2) are designed to possess two sequences, one complementary to the target (regions T1 and T2 respectively), another complementary to specific sequences on the capture probe (cp1a and cp1b). By binding to two adjacent locations on the target molecule, a “cruciform” type capture may be obtained, as disclosed by Urdea et al. in U.S. Pat. No. 5,635,352 herein incorporated by reference.

To assist the capture of target molecules in capture zones, the invention contemplates the use of re-circulation, target (nucleic acid and polypeptide) fragmentation, and a probe design for rapid hybridization of cmp and cp, using an initial rapid nucleation such as an attraction of opposite electrical charges in low salt or hydrophobic-hydrophobic interactions between molecules.

Once the capture zones have become loaded with captured target molecules via the cmps and pcmps, the presence of the captured targets within a capture zone may be detected. Various methods are contemplated for detecting captured targets. A particularly preferred method uses layered signal amplification such as described by Urdea, et al., in U.S. Pat. No. 5,635,352, herein incorporated by reference, involving optional preamplifier, an amplifier, and labeled probes.

As shown in FIGS. 1 and 2, there are a number of different methods in which signal amplification may be carried out for the nucleic acid and antigen targets. All molecules can be added sequentially as in FIG. 1A. Alternatively, some steps may be combined by forming complexes of probes and target in heating zone 21 of FIG. 3. Target can be annealed to the solid supports by using emp alone (FIG. 1B), or alternatively by using cmp and lmp (FIG. 1C), or alternatively by using emp, lmp, and PA (FIG. 1E) added together prior to passing the sample through the column. Amplifiers and labeled probes may then be added simultaneously or sequentially to the captured complex. Alternatively, lmp and PA can be combined prior to capture followed by addition of amplifier and lp (FIG. 1D).

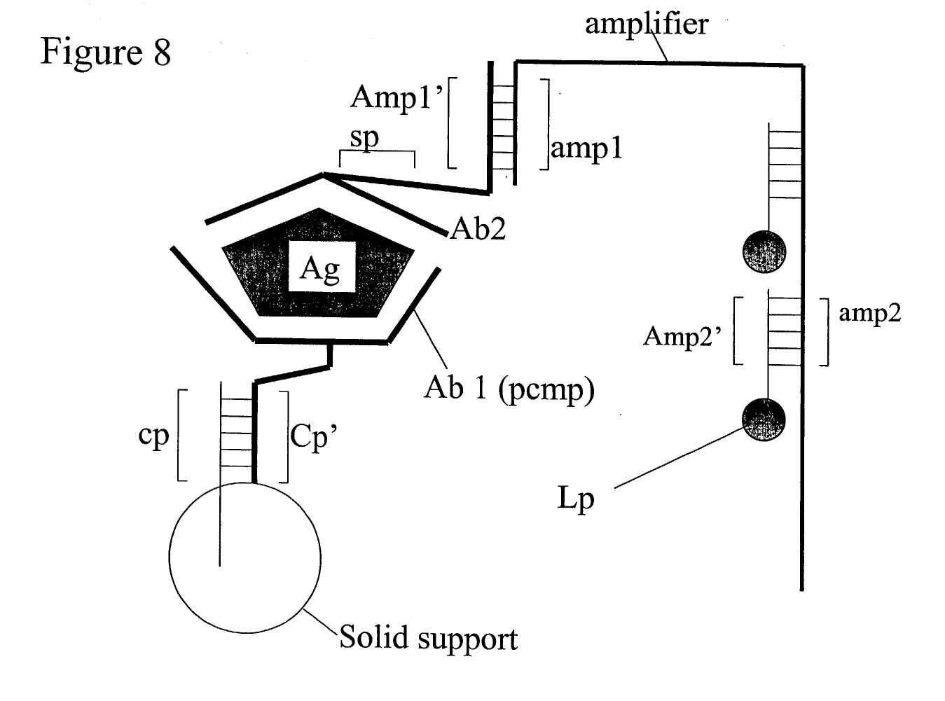

For antigen targets, the preferred method comprises capturing antigens in the antibody capture zones (section B of the column) followed by signal amplification in subsequent steps. The preferred format is the competitive immunoassay since the signal amplification steps are all generic (involving PA, amp, and lp). However, with large antigens, sandwich immunoassays can be performed. In this format, a second antibody Ab2 labeled with a sequence that is complementary to either PA, amp, or lp is added. FIG. 8 shows an example in which the second antibody is labeled with a sequence complementary to the amplifier probe. In this format the labeled Ab2 can be added before PA and re-circulated through section B or it can be added along with the PA and the mixture re-circulated through sections B and C of the column. In the subsequent steps, nucleic acids and antigens are then labeled with amplifier, then labeled probes.

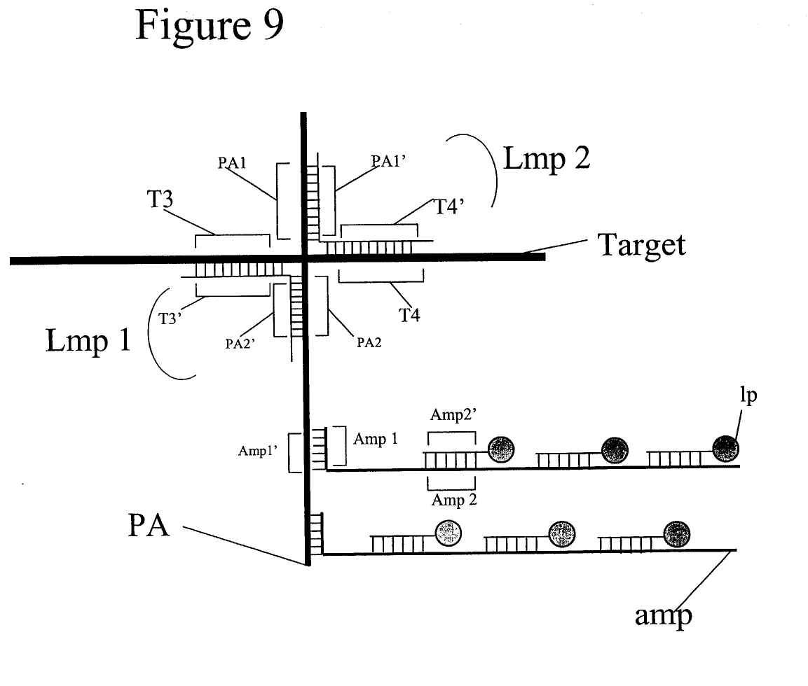

As depicted in FIG. 9, the PA is polymeric in that it possesses multiples of directly repeating sequences (Amp1′) for hybridizing to amplifier probes (via sequence Amp1). The PA is preferentially chimeric in nature, with the PA1 and PA2 sequences being PNA for facile formation of a cruciform structure with the nucleic acid target and the two PNA lmp probes. The repeating sequence of the PA (amp1′) is preferentially made of DNA. This facilitates the control of background because DNA binds poorly to solid phases in the absence of salt or in low salt buffer environments. The DNA repeat sequence of the PA also has reduced capacity for binding DNA capture probes in the low salt environment. This controls nonspecific hybridization (NSH) background. The hybridization can occur in the presence of enough salt to facilitate the approach of the negatively charged PA to the negatively charged target. A wash buffer that is substantially salt-free can be used at high temperature to reduce DNA NSB and DNA-DNA NSH because all the target-specific hybrids are PNA-DNA and are resistant to the absence of salt at high temperature.

Chimeric molecules can be made conveniently by one of two ways. One can make a PNA with a short (6 nucleotide) stretch of DNA such as the sequence dT₆. This can then serve as a site to enzymatically ligate the DNA molecule, using a complementary linker about 12 nucleotides (6 nucleotides for the DNA repeat, 6 nucleotides for the dT6) in length as disclosed by Urdea et al., in U.S. Pat. No. 5,624,802 herein incorporated by reference. The DNA molecule itself is optimally prepared by ligation of a set of oligonucleotides, a process well known in the art. Secondly, a PNA without any DNA nucleotides can be made. By attaching a functional group such as NH2, one can chemically cross-link the PNA to DNA containing COOH or aldehyde functions, or one can use SH functional group on the PNA and maleimide or haloacetimides on the DNA, which are readily prepared from DNA probes containing amines with appropriate heterobifunctional cross-linking agents (such as that available from Pierce Chemical Company). The chemical cross-linking advantageously uses a short DNA linker sequence to position the groups for efficient reaction. The chimeric product of ligation is readily purified by dual affinity chromatography (hybridization selection for the presence of the two sequences) or by gel electrophoresis or HPLC. Such methods of preparing large DNA molecules are well known in the art.

After the PAs have bound to the targets, (or with respect to antigens, the antibodies labeled with Amp1′ have bound to captured antigens), amplifier probes may be added, as pictured in FIGS. 8 and 9. The amplifier probes are preferably also chimeric molecules, with a single PNA sequence, amp1, for binding the complementary sequence amp1′ on an antibody Ab2 (FIG. 8) or on the preamplifier (FIG. 9). The binding of the amplifier probes is also carried out in sufficient salt to allow for approach of the negatively charged amplifier to the negatively charged target. The amplifiers have multiple DNA sequences, amp2 for hybridizing to the labeled probe (FIG. 9). Following amplification, washing in an substantially salt-free buffer at high temperature controls both types of backgrounds, i.e., NSH and nonspecific binding (NSB) to the solid support or to molecules bound thereto. Again, since all the target-specific hybrids are PNA-DNA, they are resistant to the absence of salt at high temperature.

The labeled probe may also be a DNA or a PNA molecule, containing a sequence amp2′, directed to bind to the repeating sequences, amp2, of the amplifier probe. Since the PA possesses multiple sites for amplifier and since the amplifier has multiple sites for labeled probe, the signal that may be generated to indicate the binding of a single target molecule at a single locus on a solid support of a capture zone may be increased as much as several hundred fold.

In a preferred embodiment, the labeled probe is one that produces light emission under appropriate conditions. For example, alkaline phosphatase may be conjugated to a nucleic acid or PNA probe amp2′ specific for the repeated sequences (amp2) of the amplifier, or in a simpler version of the assay, to a lmp that is also specific for target. Upon addition of a suitable substrate (such as a dioxetane) plus an enhancer, e.g., Lumiphos Plus from Lumigen, Inc, light is produced upon cleavage of the phosphate on the dioxetane by alkaline phosphatase.

In a preferred embodiment, the device may be placed in a sample reader having the capacity to detect light emissions emanating from any of the capture zones in a 360° circumference around the column (FIG. 4). In another embodiment, to enhance the probability of detecting light emitting events, the column may be designed in the form of an annulus or tubular doughnut (FIG. 5). In such design, a light detector may also be inserted into the center of the column. The annular design further allows any target capture event to be accessible to detection by the fact that the solid supports in any one detection zone are closer to the exterior of the column and not buried within its interior. The annular design is also particularly suited to coincidence counting, as in a scintillation counter. In either case, the light detector may be a station in a track-based instrument that docks with the column and reads individual capture zones.

Since the assay is designed to be quantitative, the use of chemiluminescence is particularly preferred since the light emission may be quantified linearly over a range spanning 5 orders of magnitude. In addition, the light output is linearly related to the number of reactive enzymes, which is linearly related to the number of captured targets, and therefore the number of targets in the sample. Thus the assay is rigorously quantitative.

### EXAMPLE 1

This example explains how to design universal sequences for the assay. First one generates sequences for signal amplification; then one generates capture probe and capture mediator sequences. One generates sequences randomly by computer having a certain range of Tm values. One then selects sequences with the following characteristics: negligible secondary structure for efficient hybridization and limited runs of purines or pyrimidines, and sometimes a fixed length. The Tm of PNA-DNA hybrids was calculated with the following empirically determined coefficients in Table I.

TABLE I

| | | | |
|---|---|---|---|
| Number | Coefficient | Delta H | delta S |

| | | | |
|---|---|---|---|
| 1 | AA | −8.4 | −22.5 |
| 2 | AC | −8.6 | −21.6 |
| 3 | AG | −6.1 | −14.3 |
| 4 | AT | −6.5 | −16.3 |
| 5 | CA | −8.6 | −22.1 |
| 6 | CC | −6.7 | −15.8 |
| 7 | CG | −10.1 | −25.9 |
| 8 | CT | −6.1 | −15.1 |
| 9 | GA | −7.7 | −18.0 |
| 10 | GC | −11.1 | −28.4 |
| 11 | GG | −6.7 | −13.0 |
| 12 | GT | −8.6 | −21.3 |
| 13 | TA | −6.3 | −19.2 |
| 14 | TC | −7.7 | −21.2 |
| 15 | TG | −8.6 | −22.7 |
| 16 | TT | −8.4 | −23.8 |
| 17 | Lys | −0.25 | 1.4 |
| 18 | Initiation | 0 | -5.9 |

The first 16 entries are the base-stacking enthalpies in Kcal/mole and the base-stacking entropies in entropy units. The entries in row 17 are enthalpies and entropies for the addition of each lysine residue to a PNA chain. The entries in row 18 are for helix initiation. The Tm is calculated as:

Tm=Enthalpy/((Entropy/1000)-0.001987* lnPo)-273.15, where “Enthalpy” is the sum of the individual enthalpies for the base stacks that make up the sequence; “Entropy/1000” is the sum of all of the individual entropies in entropy units divided by 1,000 to convert to the same scale as the enthalpies; 0.001987 is the universal gas constant; lnPo is the natural logarithm of the initial probe concentration (which is in vast molar excess over the target); and 273.15 is used to convert temperature in degrees K to temperatures in degrees C.

One may also select sequences with limited base compositional asymmetry, which is defined as the MAX−MIN, where MAX=number of the most frequently occurring base and MIN is the number of the least frequently occurring base. For example, Table II lists a set of 12 mers, containing 4 lysines, with negligible secondary structure, no runs of purines or pyrimidines longer than 3, a maximum base-compositional asymmetry of 3, and a PNA-DNA Tm of 65-70° at 1 nM. For simplicity the four lysines are not shown with the core sequence.

TABLE II

| Seq | | | | Seq | |
|---|---|---|---|---|---|
| No. | PNA sequence | Asymmetry | Tm | No. | DNA complement |
| 1 | GGCACTACACGG | 3.00 | 69.8 | 21 | CCGTGTAGTGCC |
| 2 | ATCGGTGACGCT | 2.00 | 69.5 | 22 | AGCGTCACCGAT |
| 3 | GACGGCGATGTA | 3.00 | 69.2 | 23 | TACATCGCCGTC |
| 4 | ATGACGGTTGAC | 2.00 | 69 | 24 | GTCAACCGTCAT |
| 5 | ACGCAATGTGGT | 2.00 | 68.4 | 25 | ACCACATTGCGT |
| 6 | ACGACACTGGTT | 0.00 | 68.3 | 26 | AACCAGTGTCGT |
| 7 | CGATGCCGACAA | 3.00 | 68.2 | 27 | TTGTCGGCATCG |
| 8 | CCGCAGCATTGG | 2.00 | 67.8 | 28 | CCAATGCTGCGG |
| 9 | TTGGCACGCACT | 2.00 | 67.5 | 29 | AGTGCGTGCCAA |
| 10 | TTGCCACGCCGA | 3.00 | 67.5 | 30 | TCGGCGTGGCAA |
| 11 | CGGCTATGGCAA | 2.00 | 67.1 | 31 | TTGCCATAGCCG |
| 12 | CGACTGATGTGC | 2.00 | 67.1 | 32 | GCACATCAGTCG |
| 13 | TACGATGGCAGT | 2.00 | 67 | 33 | ACTGCCATCGTA |
| 14 | CCGTGGTCAACA | 2.00 | 66.9 | 34 | TGTTGACCACGG |
| 15 | CTATGCGACGGT | 2.00 | 66.9 | 35 | ACCGTCGCATAG |
| 16 | TCGGCTGTCGCA | 3.00 | 66.6 | 36 | TGCGACAGCCGA |
| 17 | TAACGGTGTCGG | 3.00 | 66.3 | 37 | CCGACACCGTTA |
| 18 | CACGATAACGGC | 3.00 | 66.3 | 38 | GCCGTTATCGTG |
| 19 | CCATTGCGGCTG | 3.00 | 66.2 | 39 | CAGCCGCAATGG |
| 20 | CAACAGCATCGG | 3.00 | 66.1 | 40 | CCGATGCTGTTG |

Because the invention uses a low salt environment for signal amplification and high temperature virtually salt-free washes, one does not have to screen amplification sequences for homology to non-targeted sequences known to be present in the sample. The typical DNA-DNA 12 mer has a Tm of about 0° at 1 nM in 1 mM salt. In addition, the rate of hybridization of DNA to DNA is negligible at high temperature in low salt. In a one to five minute reaction, binding of PNA to DNA is strongly favored both kinetically and thermodynamically over binding of DNA to DNA and RNA to DNA. The high temperature, low salt washes further favor PNA-DNA over DNA-DNA hybridization.

From the pool of sequences generated, one selects sets of 3 sequences and their complements to be the sequences of Table III:

TABLE III

| Probe | Complement |
|---|---|
| Universal label mediator | Preamplifier unique sequence (PA1) |
| Preamplifier repeat sequence (PA2) | Amplifier unique sequence (amp1) |
| Amplifier repeat sequence (amp2) | Labeled probe |

To ensure efficient amplification, one chooses sequences that have negligible homology with any member of the set except the perfect complement. An example of such a set in which each member cannot form even a 4 mer hybrid with any other member except its perfect complement is shown in the Table below. Software such as Hybsimulator™, which is commercially available from Advanced Gene Computing Technologies, Inc., can be used to select sequences with minimal probe-probe interactions, such as displayed in Table IV.

TABLE IV

| | | |
|---|---|---|
| Seq ID No. 1 | GGCACTACACGG | PNA amplifier unique sequence |
| Seq ID No. 2 | ATCGGTGACGCT | PNA label mediator |
| Seq ID No. 3 | GACGGCGATGTA | PNA labeled probe |
| Seq ID No. 21 | CCGTGTAGTGCC | DNA preamplifier repeat |
| Seq ID No. 22 | AGCGTCACCGAT | DNA preamplifier unique sequence |
| Seq ID No. 23 | TACATCGCCGTC | DNA amplifier repeat |

Several different unique sets can be generated and amplification molecules constructed by ligation. In this way, the set most useful in probe design can be chosen for each project.

Spacers can be used in between sequences that are repeated in order to avoid creating new sequence information at the junctions, which create possibilities for new cross-hybridization. For example the preamplifier could contain trimeric repeats CCGTGTAGTGCCZZCCGTGTAGTGCCZZCCGTGTAGTGCC (SEQ ID NO. 41) rather than CCGTGTAGTGCCCCGTGTAGTGCCCCGTGTAGTGCC (SEQ ID NO. 42), where Z is a hydrophilic spacer such as triethyleneglycol.

Universal capture probe and universal capture mediator sequences are then generated similarly. The additional requirements are that the capture probe sequences must not have significant homology to the amplification sequences in Table IV and the capture mediator tails must have negligible homology with any capture probe other than their perfect complement. An example of a set of 7 such sequences is given in Table V.

TABLE V

| Seq | DNA | Seq | PNA |
|---|---|---|---|
| No. | Capture Probe | No. | capture mediator |
| 43 | TGTTTAGCCCTG | 50 | CAGGGCTAAACA |
| 44 | CAATGCTGCTCG | 51 | CGAGCAGCATTG |
| 45 | TTCTGGGTCCTA | 52 | TAGGACCCAGAA |
| 46 | CCCATTCAAGTA | 53 | TACTTGAATGGG |
| 47 | AGAACTCCACAG | 55 | CTGTGGAGTTCT |
| 48 | CTTTACCTTGCT | 55 | AGCAAGGTAAAG |
| 49 | AGCAAGCCAACT | 56 | AGTTGGCTTGCT |

A large number of these sequences are generated and synthesized. They are then screened against each other empirically for lack of cross-hybridization with each other and with the amplification sequences of Table IV. This screen is best done using conditions that quantitatively amplify the signals from even weak interactions. This is best done using concentrations of capture mediators several orders of magnitude higher (such as >1 μM) than what is used in the assay (<1 nM) and using reduced stringency compared to assay conditions. For example, stringency can be reduced by adding salt to make DNA-DNA interactions more detectable (even though the assay will be done in essentially a salt-free environment). The most sensitive and quantitative readout for direct detection of probe-probe interactions is the use of dioxetane chemiluminescence in a microtiter tray using commercially available luminometers. As an example, biotinylated capture mediator sequences (such as those in Table V) at 10 μM (in the presence of 100 μM carrier DNA to reduce NSB) would be screened for hybridization at room temperature in 1 M Na+ against the different capture probe sequences. Each capture mediator is tested against each capture probe in the microtiter dish. Hybridization is detected with streptavidin-alkaline phosphatase and dioxetane. Direct interaction of amplification sequences with capture probe sequences is done without using capture mediator probes. Preamplifier, amplifier, and alkaline phosphatase-labeled probe are added sequentially at room temperature in 1M Na+ to the wells containing a very high concentration of each capture probe (typically 200 nM is as high as practically possible). The highest practical concentration of amplification molecules would be used. Too high a concentration will produce so much nonspecific binding that specific but weak hybridization signals will be obscured.

The least cross-reactive capture probe and capture mediator sequences are chosen. The capture probe sequences are assigned to the different capture zones. The complementary sequences are the tails to be added to the target-specific sequences of the capture mediator probes.

### EXAMPLE 2

This example explains how to select targets and how to design target-specific mediator probes. The first decision is always the appropriate choice of the target molecules for the multiplex panel under consideration. The best markers to be used to detect and stage disease and assess response to therapy are in a rapid state of flux due to the explosion of information from genomics and proteomics. Some analyses must be done by immunoassay and some must be done by probe assay. However, many analytes can be detected by either method. In this case, one must first decide between immuno-markers and nucleic acid sequence markers. The choice will depend on the titer of the different targets, the availability of suitable antibodies, the availability of nucleic acid sequence information. In some cases, both antigens and nucleic acid markers will be used. This is useful for confirming positives.

For immuno-markers, antibodies are selected by methods well-known in the art for their ability to detect the highly conserved target eptitope of the target organism and to not detect any of the many possible epitopes of bystander organisms that happen to be in the sample. A panel of possible cross-reacting molecules is assembled for the screen. After completing the antibody screen and selecting a suitable antibody with the right Kd and specificity, one conjugates a universal capture mediator sequence to it (preferably through the Fc region) and binds it in the appropriate zone of section B of the column. Ideally, the column solid support is blocked to reduce nonspecific binding of the antibodies to the wrong zones. This blocking of the surface also reduces the loss of antibody activity upon binding to the surface. Hybridization is used to direct the antibody to the appropriate zone and expose the combining site for antigen recognition.

Assays requiring a sandwich immunoassay format require a second antibody that will be labeled with one of three possible sequences (label mediator, preamplifer repeat or amplifier repeat), depending on the level of signal amplification needed for detection, as explained above. Competitive immunoassays require that the antigen be labeled with one of the same three sequences. This molecule is included in the sample pre-preparation buffer or is added to the sample pre-preparation buffer by the user.

For probe targets, methods well-known in the art (e.g. Omiga Software from Oxford Molecular) would be used to align sequences from the literature (plus any sequences determined by the developer of the assay) and choose the most conserved sequences. These conserved sequences are then checked for uniqueness by doing “Blast”—type searches of all the available databases. The sequences that are both unique and conserved are then introduced into programs such as HYBsimulator™ for design of possible probes with minimal secondary structure and the correct Tm, using Table II above. Finally the probes are computer-screened for cross-hybridization to each other and the ones with minimal cross-hybridization chosen. The probes are synthesized and checked for cross-reactivity empirically and the best ones selected. Then the probes are screened empirically for appropriate inclusivity and exclusivity using appropriate panels of the targets and control nucleic acids. The term “inclusivity” refers to approximately equal (generally less than twofold difference) hybridization to all members of the target panel. For HCV, this means hybridization to representative sequences from all known genotypes (this panel must be updated as new genotypes are discovered). The term “exclusivity” refers to no detectable hybridization to all members of the control panel, which would include human sequences and viral sequences such as the other members of the hepatitis panel and other viruses commonly found in blood samples.

The final probe set is chosen as the most conserved and unique and the most inclusive and exclusive. For HCV, probes would be designed to the 5′ untranslated region or the 3′ untranslated region or to both. Examples of probe sequences with 4 lysines are given in Table VI in the highly conserved 5′ untranslated region meeting the criteria of minimal secondary structure, a Tm greater than 65 degrees in 1 mM Na+ and 1 nM probe concentration.

TABLE VI

| Seq No. | Probe # | PNA Sequence | Tm |
|---|---|---|---|
| 57 | HCV1 | TGCACGGTCTACGAG | 76.1 |
| 58 | HCV2 | CTCGCAAGCACCCTATCA | 76.7 |
| 59 | HCV3 | GGCAGTACCACAAGG | 80.3 |
| 60 | HCV4 | CCGGTTCCGCAGAC | 77.1 |
| 61 | HCV5 | CTATGGCTCTTCCGG | 71.3 |
| 62 | HCV6 | TACTAACGCCATGG | 66.5 |
| 63 | HCV7 | GCGTGAAGACAGTAG | 75.9 |

The next step is the assignment of each probe to be either a capture mediator or a label mediator. This depends on the probe-probe cross-hybridization potential and the potential to form a secondary structure between the probe sequence and the universal sequence.

The cross-hybridization potential can be measured with HYBsimulator using the “stacker” Excel program. Table VII gives the free energies of interaction among the seven probes of Table VI at 25°.

TABLE VII

| # | 57 | 58 | 59 | 60 | 61 | 62 | 63 |
|---|---|---|---|---|---|---|---|
| 57 | −5.1 | −4.7 | −3.7 | −6.2 | −4.1 | −3.3 | −5.4 |
| 58 | −4.7 | −4.6 | −3.0 | −4.7 | −6.8 | −3.3 | −4.5 |
| 59 | −3.7 | −3.0 | −3.7 | −5.6 | −6.2 | −4.7 | −2.9 |
| 60 | −6.2 | −4.7 | −5.6 | −8.1 | −8.2 | −3.0 | −4.5 |
| 61 | −4.1 | −6.8 | −6.2 | −8.2 | −5.9 | −6.2 | −5.0 |
| 62 | −3.3 | −3.3 | −4.7 | −3.0 | −6.2 | −7.2 | −5.9 |
| 63 | −5.4 | −4.5 | −2.9 | −4.5 | −5.0 | −5.9 | −2.9 |

These are more conveniently turned into equilibrium association constants at 60°, as shown in Table VIII.

TABLE VIII

| | 57 | 58 | 59 | 60 | 61 | 62 | 63 |
|---|---|---|---|---|---|---|---|
| 57 | 2,259 | 1,198 | 268 | 10,880 | 455 | 140 | 3,610 |
| 58 | 1,198 | 1,014 | 88 | 1,253 | 28,621 | 144 | 872 |
| 59 | 268 | 88 | 276 | 4,810 | 12,278 | 1,234 | 77 |
| 60 | 10,880 | 1,253 | 4,810 | 207,261 | 241,076 | 88 | 872 |
| 61 | 455 | 28,621 | 12,278 | 241,076 | 7,570 | 12,094 | 1,856 |
| 62 | 140 | 144 | 1,234 | 88 | 12,094 | 53,186 | 7,921 |
| 63 | 3,610 | 872 | 77 | 872 | 1,856 | 7,921 | 83 |

The strongest interactions are highlighted. These are all very weak interactions, compared to probe-target interactions, which are greater than 10¹⁰. Label mediator-capture mediator hybridization does not cause significant background in the present invention due to the removal of mediator probes not bound to target. Nevertheless, for applications requiring extreme sensitivity, it is best to minimize these interactions.

Probe 60 and probe 62 interact weakly with themselves. As long as they are weak, these interactions are of little consequence. Probe 61 interacts with probes 58 and 60. One could eliminate probe 61 from the set or could make probes 58, 60, and 61 all capture mediator or all label mediator probes.

Next, all probe sequences are tested for secondary structure with the label mediator sequence. The results are shown in Table IX. Two hydrophilic spacers (denoted “Z”) are inserted between the probe sequence and the universal sequence. This breaks up any secondary structures created by new sequence information at the junction. The loop Tm and delta G are approximate, since they are calculated for DNA-DNA internal structures (values for PNA-PNA are not available). One can assume that the PNA secondary structures will be stronger than the DNA-DNA since double-stranded PNA hybrids have higher Tm values than PNA-DNA hybrids, which are higher than DNA-DNA hybrids in physiological salt.

TABLE IX

| Seq | | | Loop |
|---|---|---|---|
| No. | Concatenated Sequence | Delta G | Tm |
| 64 | TGCACGGTCTACGAGZZATCGGTGACGCT | −0.7 | 42 |
| 65 | CTCGCAAGCACCCTATCAZZATCGGTGACGCT | −1.8 | 58 |
| 66 | GGCAGTACCACAAGGZZATCGGTGACGCT | 0.1 | 23 |
| 67 | CCGGTTCCGCAGACZZATCGGTGACGCT | −2.5 | 71 |
| 68 | CTATGGCTCTTCCGGZZATCGGTGACGCT | −2.3 | 67 |
| 69 | TACTAACGCCATGGZZATCGGTGACGCT | 0.7 | 0 |
| 70 | GCGTGAAGACAGTAGZZATCGGTGACGCT | −0.9 | 39 |

Probes 65, 67, and 68 are least suitable as label mediators because the loop Tm of the secondary structures are close to the incubation temperatures. Probes 66 and 69 are most suitable as label mediators. Probes 64 and 70 are likely to be suitable as label mediator probes at 60-65°. The label mediator probes do not have to be screened for cross-hybridization potential to the universal capture probe sequences since free mediators are removed prior to entry to section C. For more sensitive work this screen could be included and would result in the rejection of additional probes.

A large number of different capture mediator universal sequences can be used. One tests each possible sequence for suitability with the probe sequences. An example of an appropriate universal sequence to use is given in Table X.

TABLE X

| Seq | | | Loop |
|---|---|---|---|
| No. | Capture Mediator Sequence | Delta G | Tm |
| 71 | TGCACGGTCTACGAGZZCAGGGCTAAACA | 0.8 | — |
| 72 | CTCGCAAGCACCCTATCAZZCAGGGCTAAACA | −3.7 | 74 |
| 73 | GGCAGTACCACAAGGZZCAGGGCTAAACA | 1.2 | — |
| 74 | CCGGTTCCGCAGACZZCAGGGCTAAACA | 0.9 | — |
| 75 | CTATGGCTCTTCCGGZZCAGGGCTAAACA | 1 | — |
| 76 | TACTAACGCCATGGZZCAGGGCTAAACA | −2 | 63 |
| 77 | GCGTGAAGACAGTAGZZCAGGGCTAAACA | 1.5 | — |

In this set, only probes 72 and 76 are unsuitable as capture mediators at 60°. The best probe set with these universal overhangs is shown in Table XI. Probes 72 and 77 are not used. Additional capture mediator and label mediator probes may be found in the 3′ untranslated region of HCV. Target fragmentation during sample pre-preparation or sample preparation on the column improves the efficiency of probing the different regions of the virus.

TABLE XI

| Seq | | | | |
|---|---|---|---|---|
| No. | Mediator Type | Sequence | Delta G | Loop Tm |
| 71 | Capture mediator | TGCACGGTCTACGAGZZCAGGGCTAAACA | 0.8 | — |
| 66 | Label mediator | GGCAGTACCACAAGGZZATCGGTGACGCT | 0.1 | 23 |
| 74 | Capture mediator | CCGGTTCCGCAGACZZCAGGGCTAAACA | 0.9 | — |
| 75 | Capture mediator | CTATGGCTCTTCCGGZZCAGGGCTAAACA | 1 | — |
| 69 | Label mediator | TACTAACGCCATGGZZATCGGTGACGCT | 0.7 | 0 |

Since the target will be fragmented, another consideration is that capture mediator and label mediator be as close as possible on the target sequence. This is met by the above probe set. Yet another consideration is to have more than one label mediator and more than one capture mediator in the final probe set because there may be polymorphisms yet to be discovered in one of the targeted sequences.

Some probes are suitable as capture mediators or label mediators and can be synthesized both ways. Empirical testing is then used to select the final probe sets for the panels.

An entire panel may be constructed similarly by designing probes for each target. Capture mediator-label mediator interactions are screened on computer as one large pool against another large pool of possible sequences. Then they are made and screened empirically individually at first and then finally as pools of sequences.

### EXAMPLE 3

In one method of using the invention, cmps and pcmps are preloaded into various zones in the column using the appropriate universal sequences and channeling the cmps directly from section B to section C without passing through zone 21. (Alternatively, cmps are mixed with sample and sample buffer). In preparing the sample, sample preparation buffer may contain in addition to cmps, labeled competitor antigens, PA and lmps. Alternatively, (except for the labeled competitor antigens) the PA and lmps may be directed through the column after sample has been processed through the column as previously discussed.

The samples are denatured with detergents and specific enzymes (e.g., bead-immobilized enzymes). The sample mixture is then passed over the column with the immobilized enzymes and denaturants being removed as the sample mixture passes through section A. The sample preparation for antigen targets is completed in section A, optimally by re-circulation. After completing sample preparation in section A, antigens and labeled competitor antigens are captured in section B by repeated re-circulation.

The sample is then directed to heating element 21 by properly positioning valve 15b. Sample preparation for nucleic acids is completed on the column in zone 21 following the denaturation of the targets. While the sample mixture is incubated in the denaturation/high temperature zone 21, the target nucleic acid molecules are annealed with cmps (and signal amplifying probes i.e., lmp and optionally PA). The sample may then be re-circulated through section C as many times as desired for capture of the target/probe complex to specific capture zones.

Instead of capturing nucleic acid targets through a single pre-immobilized capture probe (as with antigen capture), many cmps (as shown in FIG. 6) may hybridize to the nucleic acid target molecule in heating zone 21, thereby providing a greater probability of capturing the nucleic acid targets as they pass through the capture zones.

After capture of antigens and nucleic acids in the appropriate zones, signal amplification molecules, if they were not already provided in the sample preparation mixture, are added through port 26. First, PA, and optionally antibodies containing amp1′ sequence are added and re-circulated through the column. Labeled antibodies are added through 26. The PA, amplifier, labeled probe in succession are added through port 26 and re-circulated through sections B and C of the column.

In another embodiment, no preamplifier or amplifier probes are used. Rather, target-specific nucleic acid lmps and/or antibodies having the amp2 sequence are added along with a labeled probe having the amp2′ sequence. In a preferred embodiment, the label of the labeled probe is alkaline phosphatase.

Once the label probes are added, the column is washed and then incubated with a solution containing a light-emitting molecule such as dioxetane. Upon the dioxetane encountering a probe labeled with alkaline phosphatase, the dioxetane is cleaved, resulting in the emission of visible light. Optimally the solid phase is designed to bind dephosphorylated dioxetanes prior to emission for a more focused emission of light. For example, nylon is well-known in the art to possess this property and could be coated on the surface of beads.

Advantages of the method described for Example 1 are that antibodies Ab1 and Ab2 are never exposed to heat, denaturing detergents or proteases. Only the antigens are exposed to denaturing agents, while only nucleic acid targets are exposed to extremes of both heat and denaturing agents. Use of such a protocol favors multiplex assays wherein a panel of immunoassays are to be performed along with nucleic acid probe tests.

### EXAMPLE 4

An example of a panel for screening samples for disease states is a panel to screen whole blood for HIV infection and at the same time provide drug monitoring information and a status report of the patient's immune competence. By “HIV” is meant members of both clade M and dade O (“outliers”). A zone specific for HIV 2 could be included as well. The term “drug monitoring” is meant to include tests that determine patient compliance with drug regimens as well as tests that provide indication of drug overdose or underdose, or adverse reactions to drugs.

In this example, a sample comprising whole blood is treated with detergents and enzymes as described above. Also included in the sample preparation buffer is an internal quantification standard for viral load such as unlabeled simian immune deficiency virus (SIV) RNA. The use of such a standard will allow evaluation of capture and labeling efficiency as well as quantification of sample-derived HIV viral load. A standard curve spanning the useful quantification range can be prepared by capturing different SIV sequences in different capture zones. A particularly useful quantification range would comprise a group of different SIV sequences from 10² molecules/mL up to 10⁷ molecules/mL.

The HIV RNA suspected to be in the sample, along with the SIV standard, is fragmented to an average size of about 100-300 nucleotides and hybridized with one or more cmp and lmp. Fragmentation of target molecules (and sample nucleic acids and proteins) is crucial, since numerous sites within the virus are to be probed and the viral load is often very low in patients undergoing triple or quadruple therapy. Fragmentation allows each segment of the viral genome to contribute signal to each capture zone. It further reduces sample viscosity (due to high molecular weight sample DNA) and improves hybridization and capture efficiency.

The sample is passed through the column wherein the targets (unknowns and standards) are captured in specified zones. With respect to the sample-derived HIV, the column may be designed to capture (1) conserved sequences within HIV gag or HIV pol RNA for determining viral load, (2) HIV envelope RNA to determine genotype of the viral strain or serotype, (3) selected HIV pol RNA sequences for drug resistance so that a proper therapeutic regimen may be initiated or continued, (4) cellular antigens that indicate immune status e.g., CD4+, CD8+, and IL-2, (5) various molecules derived from opportunistic infections such as CMV, Kaposi's sarcoma, and pneumocystis, and (6) various analytes for drug monitoring.

After passing the sample through the column, signal amplification molecules are added, the column is washed and placed in a light detection device for reading positive light signals emanating from the capture zones.

The drug metabolite immunoassays may be carried out in the competitive mode described above. The capture antibodies would be pre-loaded into zones in section B. The sample pre-preparation buffer would contain selected drug metabolites labeled with a sequence complementary to either PA, amp, or lp, depending on the expected titer. As the sample is re-circulated through the column the sample-derived antigen and the labeled antigen would compete for binding to the antibody. The signal will be reduced in direct proportion to the amount of antigen present in the sample. A similar competitive assay can be used as a calibrator. A standard curve can be used to translate the light output into molecules of drug metabolite per milliliter. A format with a capture efficiency of greater than 90% would have the advantage that a single calibration curve could be constructed for all analytes (probe and antibody) that directly translates quantitative light output into number of captured molecules (drugs, peptide antigens, nucleic acid molecules).

Drug-resistance would optimally be determined as follows. For each test site, two capture mediator probes, w to recognize the wild type sequence w′ and m to recognize the mutant sequence m′, would be designed with different universal tails to bind to different zones in the column. The other two allelic sequences y and z, not found in nature, would be tailless and could be added in excess, typically 10-100 fold. Their function is to compete with mismatched hybrids and thus reduce background. If only wild type sequence w′ is present, then the wild type probe w will bind strongly to its locus and the three mismatches, m, y, and z will compete with each other for binding to the wild type sequence. Since the binding constants of m, y, and z are similar and much less than w, the two nonsense mismatches y and z will bind to more molecules than m due to their higher molar concentration. This lower level of binding with the mismatched capture mediator probe m will thus lower the background present in that capture zone and allow a more accurate estimate of the mutant and wild type populations at that test locus.

When both wild type and mutant sequences are present, due to their much higher binding constants, capture mediators w and m will successfully compete with the tailless sequences y and z and will capture targets in their respective zones. Upon labeling, the ratio of mutant/wild type sequence is determined by the ratio of light emitted from the two capture zones. When mutant sequence is unambiguously detected, it is time to change the drug treatment regimen. Clearly, the more sensitive and specific the assay is, the earlier the detection of drug resistance. The use of a single, very short (fixed Tm) PNA cmp for each locus, e.g. 8-14 bases, is contemplated for the target-specific portion of the capture mediator probes, to maximize the specificity. Typically, 15-20 degree changes in Tm occur when a probe of that size is hybridized to a sequence with a single nucleotide change. This Tm difference typically translates into a 100-1,000 fold change in binding constant.

Alternatively, two short (6-10 bases) cruciform emp (i.e., a pair of allelic probes comprising the wild type and mutant sequences) can be used with the second probe for cruciform capture comprising an adjacent conserved sequence. The label mediator probes used for detection will typically be 15-18 bases. Together with the capture mediator, this will provide 23-32 bases of information to be sure that unique sequences in the sample potentially containing large amounts of human DNA are being correctly targeted.

Rather than direct all the different mutations to different zones on the column, groups of mutations that confer resistance to a drug, or a group of drugs, can be monitored in each zone. The presence of any one mutation in a zone signifies emerging development of resistance to the drug.

Assays that use target amplification have two distinct disadvantages in quantifying mutant and wild type populations, namely unequal amplification of the sequences and unequal hybridization efficiency of the DNA probes in high salt to the different sequences, which may have very different accessibility to the probes (as well as different Tm values and different rate of binding in rapid assays). The present invention circumvents these problems by not amplifying the target and by using PNA probes at high temperatures in the absence of salt to achieve complete and thus equal hybridization efficiency to the two allelic sequences.

### EXAMPLE 5

This is a variant of example 2 in which additional up front sample preparation may be carried out to obtain results that directly correspond to assays currently in use. In this example, blood is first fractionated into plasma and a cell pellet. The cell pellet is reacted with solid phase containing anti-CD4+, anti-CD8+, anti-IL-2. The particles are washed, and cells binding solid phase are lysed and combined with the plasma in the sample for analysis as described above. This panel may be designed for both drug monitoring in plasma and viral load testing at the genetic level in plasma, while also specifically measuring the level of cells containing key immune status markers. Thus, the invention is able to provide testing at three levels as compared to drug monitoring assays, viral load assays, and flow cytometric assays for CD4+ cells that must be conducted separately using existing technology.

### EXAMPLE 6

A screening panel for hepatitis viruses may be established wherein whole blood samples are treated in sample buffer as described above. The prepared sample is added to the column wherein capture zones are specific for testing hepatitis viruses A, B, C, D, E, G, and subtypes thereof (i.e., HAV, HBV, HCV, HDV, HEV, and HGV). Such zones may be specific for either polypeptide targets (section B) or nucleic acid targets (section C), or both. Other capture zones may be specific for metabolic markers for evaluating the health of the patient such as indicators of liver damage such as alanine aminotransferase (ALT) or aspartate aminotransferase (AST). As with HIV, the capture zones may be directed to determining viral load, genotype of the virus, and markers for drug resistance.

Specific assays for following hepatitis C virus (HCV) and hepatitis B virus (HBV) during therapy can also be envisioned that also perform drug monitoring tests to detect drug compliance, overdose, underdose, and adverse reactions.

As noted above, the preferred RNA target for HCV is the 5′ untranslated region where there are several short regions of conserved sequence and the 3′ untranslated region, where there are about 100 conserved nucleotides (4 to 6 probes). The conserved sequences on the HBV genome are widely scattered on the 3.2 kb genome.

### EXAMPLE 7

A blood screening panel would consist of probes for HIV, HCV, HBV, HTLV I, and possibly hepatitis G virus (HGV). A combination of antigen (mostly internal) and nucleic acid markers would be used for an internal check on the results. The assay would use whole blood. Direct assay of the antigens and nucleic acids allows tainted blood to be detected more efficiently than current immunoassays that measure antibodies to the viruses. Consequently tainted blood samples can be detected before a patient seroconverts.

### EXAMPLE 8

A screening panel for enteric pathogens may be established wherein the capture zones in either section B or C, or both B and C, are designed individually to screen for such organisms as Salmonella sp., enterotoxigenic E. coli strains, Shigella sp., Campylobacter sp., Listeria sp., Yersinia sp., and Vibrio cholerae. Various immunoassays can further be used to assess the current status of the patient. The protocol is equivalent to any of the foregoing examples.

### EXAMPLE 9

Sexually transmitted diseases may be tested via a panel designed to test for such agents as HIV, N. gonorrhea, Trichomonas, Chlamydia, Candida, T. pallidum, the most clinically relevant subtypes of human papilloma virus (HPV), and herpes simplex virus (HSV). The protocol for such testing is equivalent to any of the foregoing examples.

### EXAMPLE 10

Water samples may be tested wherein a high volume sample (typically 1,000 liters) is concentrated on a submicron filter and resuspended in a few milliliters. Following such reduction in volume, the sample is treated with sample pre-preparation buffer and applied to a column containing a panel of capture zones specific for total coliforms as a general measure of fecal contamination and chlorine-resistant organisms such as Giardia lamblia, Cryptosporidium parvum, Entamoeba histolytica. Antigen targets suitable for the present invention have already been identified and incorporated into a commercial product, the Triage™ Parasite panel, available from Biosite Diagnostics, Inc. (San Diego, Calif.).

### EXAMPLE 11

In a manner similar to those described above, stool samples may be tested for a variety of targets including enteric pathogens, metabolites associated with various disorders, presence of blood, etc.

### EXAMPLE 12

A blood poisoning panel may comprise various antigen markers for septic shock, immune status, and broad classes of organisms (eubacterial probes, fingal probes, protozoan probes and viral probes).

### EXAMPLE 13

A meningitis panel might consist of probes for distinguishing viral and bacterial meningitis as well as various immunological markers.

### EXAMPLE 14

A respiratory disease panel might consist of probes for all members of the Mtb complex including M. africanum, M. bovis, M. microti, M. tuberculosis, as well as specific tests for resistance to the major classes of anti-TB drugs. Immunoassays to detect antigens associated with Mtb disease would also be included. A number of other respiratory pathogens could be included in an initial screen such as numerous agents that cause pneumonia and respiratory syncytial virus (RSV).

### EXAMPLE 15

Heart disease panels can be constructed that either screen for genetic markers of predisposition, as these are discovered by cardiogenomics research, or markers of impending disease or that measure markers of disease progression, including the heart attack markers, CK-MB, Troponin I, myoglobin, etc.

### EXAMPLE 16

Numerous cancer panels can be constructed. For example, for breast cancer monitoring there could be estrogen receptor (ER) mRNA, progesterone receptor (PR) mRNA and control mRNAs (housekeeping genes such as ubiquitin or glyceraldehyde phosphate dehydrogenase), which serve as a cell counting assay. There would be zones to measure her2/neu gene amplification and other key marker gene amplifications, and one or more generic chromosome 17 markers as well as one or more single copy gene/genome calibrators (which also serve as a cell counting assay). The ER and PR message assays could instead be immunoassays for the proteins in the breast biopsies. In addition there would be drug monitoring immunoassay tests to complete the panel.

### EXAMPLE 17

The generic nature of the chemistry lends itself to a track-based instrument to automate the assays, allowing high throughput. For example, there could be a plurality of work stations having timed assays and timed intervals between stations. Assay results could be available within one hour and assay rate could be on the order of 120 samples/hour. After mixing the sample and the sample preparation reagents, and after an optional incubation, the user would add the pre-processed sample to the column and put it on the instrument. The Table XII below lists an example of discrete steps in the assay with approximate timing for each step. The time is an integral multiple of 30 seconds to maintain indexing.

TABLE XII

| Station | | Approximate |
|---|---|---|
| Number | Operation | Time (minutes) |
| 1 | Pass sample through section A | 2 |
| 2 | Re-circulate through section B | 2 |
| 3 | Heat in zone 21 | 2 |
| 4 | Incubate in zone 21 | 5 |
| 5 | Re-circulate through section C | 4 |
| 6 | Wash sections B and C | 1 |
| 7 | Re-circulate preamplifier through sections | 1 |
| | B and C | |
| 8 | Wash sections B and C | 1 |
| 9 | Re-circulate amplifier through sections | 1 |
| | B and C | |
| 10 | Wash sections B and C | 1 |
| 11 | Re-circulate labeled probe through sections | 1 |
| | B andC | |
| 12 | Wash sections B and C | 3 |
| 13 | Incubate with substrate in sections B and C | 5 |
| 14 | Measure luminescence | 1 |

The present invention has been described above with reference to preferred embodiments. Numerous technical aspects respecting use of routine laboratory procedures for preparing nucleic acid and protein samples as well as determining specific targets to which panels may be directed are either well known in the art or are easily determinable by one skilled in the medical and molecular biological arts. Thus, a detailed explanation of technical procedures and specific target antigens and nucleic acid sequences in the examples is not warranted. Moreover, it would be obvious to one of ordinary skill in the art that many additions, deletions and changes can be made without departing from the spirit and the scope of the invention as claimed below.

What is claimed is:

1. An apparatus for carrying out sample preparation and multiplex detection of panels of target nucleic acids and antigens in a sample comprising:
(a) a sample preparation zone;
(b) a multiplicity of three dimensional capture probe platforms for capturing specific classes of target molecules, said three dimensional platforms further comprising sets of platforms, said sets further being physically separated from one another, said sets further having capture probes that are specific for capturing any one specific class of said target molecule; and
(c) spacer elements for separating said sets of three dimensional capture probe platforms, said sets and spacer elements further arranged so as to allow a sample to pass in succession from one set through a spacer element to another set.

2. An apparatus according to claim 1 wherein said capture probe platforms are transparent to visible light transmission.

3. An apparatus according to claim 1 wherein said spacer elements are opaque to visible light transmission.

4. An apparatus according to claim 1 wherein said spacer elements have an outer surface that has a high reflective index.

5. An apparatus according to claim 1 wherein said capture probe platforms have attached thereto a plurality of oligonucleotides.

6. An apparatus according to claim 5 wherein said oligonucleotides have a base sequence that is unique for each of said sets of platforms.

7. An apparatus according to claim 1 wherein said panel is directed to the multiplex detection of nucleic acids and antigens derived from an infective agent selected from the group consisting of a pathogenic microorganism, a bacterium, a protozoan, a virus, a fungus, and a prion.

8. An apparatus according to claim 1 wherein said capture probes of any given set of said sets are complementary to nucleic acid probes that are themselves complementary in part to nucleic acids derived from a source selected from the group consisting of a microorganism, a bacterium, a protozoan, a virus, and a fungus.

9. An apparatus according to claim 1 wherein said capture probes of any given set of said sets are complementary to nucleic acid sequences that are themselves attached to antibodies specific for antigens derived from a source selected from the group consisting of a drug metabolite, a microorganism, a bacterium, a protozoan, a virus, and a fungus.

10. An apparatus according to claim 1 wherein said panel is directed to the multiplex detection of nucleic acids and antigens associated with a disease state selected from the group consisting of acquired immune deficiency, hepatitis, venereal disease, respiratory disease, diarrheal illness, a viral infection, a bacterial infection, a protozoan infection, heart disease and cancer.

11. An apparatus according to claim 1 wherein said capture probes of any given set of said sets are complementary to nucleic acid probes that are themselves complementary in part to nucleic acids of an organism selected from the group consisting of Salmonella sp., enterotoxigenic E. coli strains, Shigella sp., Campylobacter sp., Listeria sp., Yersinia sp., Vibrio cholerae, N. gonorrhea, Trichomonas, T. pallidum, Chlamydia, Candida, Giardia lamblia, Cryptosporidium, and total coliforms.

12. An apparatus according to claim 1 wherein said capture probes of any given set of said sets are complementary to nucleic acid sequences that are themselves attached to antibodies specific for antigens derived from an organism selected from the group consisting of Salmonella sp., enterotoxigenic E. coli strains, Shigella sp., Campylobacter sp., Listeria sp., Yersinia sp., Vibrio cholerae, N. gonorrhea, Trichomonas, T. pallidum, Chlamydia, Candida, Giardia lamblia, Cryptosporidium, and total coliforms.

13. An apparatus according to claim 1 wherein said capture probes of any given set of said sets are complementary to nucleic acid probes that are themselves complementary in part to nucleic acids of a virus selected from the group consisting of RSV, HPV, HSV, HIV, HCV, HBV, HGV, HDV, HEV, HTLV-1, and HTLV-2.

14. An apparatus according to claim 1 wherein said capture probes of any given set of said sets are complementary to nucleic acid sequences that are themselves attached to antibodies specific for antigens derived from a virus selected from the group consisting of RSV, HPV, HSV, HIV, HCV, HBV, HGV, HDV, HEV, HTLV-1 and HTLV-2.

15. An apparatus according to claim 1 wherein said panel is directed to the multiplex detection of a virus selected from the group consisting of RSV, HPV, HSV, HIV, HCV, HBV, HGV, HDV, HEV, HTLV-1 and HTLV-2.

16. An apparatus according to claim 1 wherein said panel is directed to the multiplex detection of nucleic acids and/or antigens selected from the group consisting of HIV gag RNA, HIV envelope RNA, HIV polymerase RNA, HIV encoded proteins, immune cell derived molecules, cytomegalovirus, antigens or nucleic acids derived from the presence of Kaposi's sarcoma, drug metabolites, and antigens or nucleic acids derived from pneumocystis infection.

17. An apparatus according to claim 1 wherein said panel is directed to the multiplex detection of organisms selected from the group consisting of Salmonella sp., enterotoxigenic E. coli strains, Shigella sp., Campylobacter sp., Listeria sp., Yersinia sp., and Vibrio cholerae.

18. An apparatus according to claim 1 wherein said panel is directed to the multiplex detection of a of organisms selected from the group consisting of Giardia lamblia, Cryptosporidium parvum, Entamoeba histolytica, and total coliforms.

19. An apparatus according to claim 1 wherein said panel is directed to the multiplex detection of hepatitis viruses and associated clinical markers selected from the group consisting of hepatitis A, hepatitis B, hepatitis C, hepatitis D, hepatitis E, hepatitis G, ALT, AST, and drug metabolites.

20. An apparatus according to claim 1 wherein said panel is directed to the multiplex detection of sexually transmitted diseases caused by pathogenic agents selected from the group consisting of acquired immune deficiency virus, N. gonorrhea, Trichomonas, Chlamydia, Candida, T. pallidum, HPV, and HSV.

21. An apparatus according to claim 1 wherein said panel is directed to the multiplex detection of respiratory pathogens and/or resistance of such pathogens to anti-TB drugs selected from the group consisting of pneumonia causing bacteria, RSV, M. africanum, M. bovis, M. microti, and M. tuberculosis.

22. An apparatus according to claim 1 further comprising the elements of:
(a) a inert filter zone for filtering particulate material from a sample;
(b) a porous element zone for filtering small ionic materials from a sample;
(c) a multiplicity of zones comprising enzymes for carrying out digestions of sample- derived materials;
(d) a sample temperature adjustment zone; and
(e) a multiplicity of re-circulation zones wherein a sample may be imported into said apparatus, said elements of (a), (b), (c), (d), and (e) further being connected together in any sequence while a sample may pass from one element to another and thereafter may further pass to said sets.

23. A method of carrying out multiplex detection of target nucleic acids and antigens in a sample comprising the steps of:
(a) providing a sample containing target nucleic acids and/or antigens of interest;
(b) treating said sample with a sample buffer to form a pre-processed sample;
(c) passing said pre-processed sample over an apparatus of claim 1;
(d) capturing said target nucleic acids and antigens by capture probes of said apparatus of claim 1;
(e) reacting a label with a signal probe, said signal probe having specificity for at least one other signal probe that is specific for said target of (d); and
(f) detecting said reacted label in any of said sets of said apparatus of claim 1.

24. A method of carrying out multiplex detection of target nucleic acids and antigens in a sample comprising the steps of:
(a) providing a sample containing target nucleic acids and/or antigens of interest;
(b) treating said sample with a sample buffer to form a pre-processed sample;
(c) passing said pre-processed sample over an apparatus of claim 22;
(d) capturing said target nucleic acids and antigens by capture probes of said apparatus of claim 22;
(e) reacting a label with a signal probe, said signal probe having specificity for at least one other signal probe that is specific for said target of (d); and
(f) detecting said reacted label in any of said sets of said apparatus of claim 22.

25. A method according to claim 23 for detecting a panel of pathogenic agents selected from the group consisting of Salmonella sp., enterotoxigenic E. coli strains, Shigella sp., Campylobacter sp., Listeria sp., Yersinia sp., Vibrio cholerae, N. gonorrhea, Trichomonas, Chlamydia, Candida, Giardia lamblia, Cryptosporidium, and total coliforms.

26. A method according to claim 24 for detecting a panel of pathogenic agents selected from the group consisting of Salmonella sp., enterotoxigenic E. coli strains, Shigella sp., Campylobacter sp., Listeria sp., Yersinia sp., Vibrio cholerae, N. gonorrhea, Trichomonas, Chlamydia, Candida, Giardia lamblia, Cryptosporidium, and total coliforms.

27. A method according to claim 23 for detecting a panel of disease states selected from the group consisting of acquired immune deficiency, hepatitis, venereal disease, respiratory disease, diarrheal illness, a viral infection, a bacterial infection, a protozoan infection, heart disease, and cancer.

28. A method according to claim 24 for detecting a panel of disease states selected from the group consisting of acquired immune deficiency, hepatitis, venereal disease, respiratory disease, diarrheal illness, a viral infection, a bacterial infection, a protozoan infection, heart disease, and cancer.

29. A method according to claim 23 wherein said panel is directed to the multiplex detection of a viral agent selected from the group consisting of RSV, HPV, HSV, HIV, HCV, HBV, HTLV-1, HTLV-2, HGV, hepatitis D, and hepatitis E.

30. A method according to claim 24 wherein said panel is directed to the multiplex detection of a viral agent selected from the group consisting of RSV, HPV, HSV, HIV, HCV, HBV, HTLV-1, HTLV-2, HGV, hepatitis D, and hepatitis E.

31. A method according to claim 23 wherein said panel is directed to the multiplex detection of hepatitis B associated targets selected from the group consisting of HBV viral load, HBV genotype or serotype, ALT, AST, drug resistance, and drug monitoring.

32. A method according to claim 24 wherein said panel is directed to the multiplex detection of hepatitis B associated targets selected from the group consisting of HBV viral load, HBV genotype or serotype, ALT, AST, drug resistance, and drug monitoring.

33. A method according to claim 23 wherein said panel is directed to the multiplex detection of hepatitis C associated targets selected from the group consisting of HCV viral load, HCV genotype or serotype, ALT, AST, drug resistance, and drug monitoring.

34. A method according to claim 24 wherein said panel is directed to the multiplex detection of hepatitis C associated targets selected from the group consisting of HCV viral load, HCV genotype or serotype, ALT, AST, drug resistance, and drug monitoring.

35. A method according to claim 23 wherein said panel is directed to the multiplex detection of a microorganism selected from the group consisting of Salmonella sp., enterotoxigenic E. coli strains, Shigella sp., Campylobacter sp., Listeria sp., Yersinia sp., and Vibrio cholerae.

36. A method according to claim 24 wherein said panel is directed to the multiplex detection of a microorganism selected from the group consisting of Salmonella sp., enterotoxigenic E. coli strains, Shigella sp., Campylobacter sp., Listeria sp., Yersinia sp., and Vibrio cholerae.

37. A method according to claim 23 wherein said panel is directed to the multiplex detection of a of a virus or other target molecule associated with hepatitis infection and treatment selected from the group consisting of hepatitis A, hepatitis B, hepatitis C, hepatitis D, hepatitis E, hepatitis G, ALT, AST, and hepatitis drug metabolites.

38. A method according to claim 24 wherein said panel is directed to the multiplex detection of a of a virus or other target molecule associated with hepatitis infection and treatment selected from the group consisting of hepatitis A, hepatitis B, hepatitis C, hepatitis D, hepatitis E, hepatitis G, ALT, AST, and hepatitis drug metabolites.

39. A method according to claim 23 wherein said panel is directed to the multiplex detection of a pathogenic agent selected from the group consisting of acquired immune deficiency virus, N. gonorrhea, Trichomonas, T. pallidum, Chlamydia, Candida, HPV, and HSV.

40. A method according to claim 24 wherein said panel is directed to the multiplex detection of a pathogenic agent selected from the group consisting of acquired immune deficiency virus, N. gonorrhea, Trichomonas, T. pallidum, Chlamydia, Candida, HPV, and HSV.

41. A method according to claim 23 wherein said panel is directed to the multiplex detection of a microorganism selected from the group consisting of Giardia lamblia, Cryptosporidium parvum, Entamoeba histolytica, and total coliforms.

42. A method according to claim 24 wherein said panel is directed to the multiplex detection of a microorganism selected from the group consisting of Giardia lamblia, Cryptosporidium parvum, Entamoeba histolytica, and total coliforms.

43. A method according to claim 23 wherein said panel is directed to the multiplex detection of nucleic acids and antigens derived from a source selected from the group consisting of a microorganism, a bacterium, a protozoan, a virus, and a fungus.

44. A method according to claim 24 wherein said panel is directed to the multiplex detection of nucleic acids and antigens derived from a source selected from the group consisting of a microorganism, a bacterium, a protozoan, a virus, and a fungus.

45. A method according to claim 23 wherein said panel is directed to the multiplex detection of respiratory pathogens and/or resistance of such pathogens to anti-TB drugs selected from the group consisting of a pneumonia causing bacteria, RSV, M. africanum, M. bovis, M. microti, and M. tuberculosis.

46. A method according to claim 24 wherein said panel is directed to the multiplex detection of respiratory pathogens and/or resistance of such pathogens to anti-TB drugs selected from the group consisting of a pneumonia causing bacteria, RSV, M. africanum, M. bovis, M. microti, and M. tuberculosis.

47. An apparatus according to claim 1 that is a track-based instrument to automate assays.

48. A method according to claim 23 wherein the apparatus of claim 1 is a track-based instrument to automate assays.

49. A method according to claim 24 wherein the apparatus of claim 22 is a track-based instrument to automate assays.

50. An apparatus for carrying out sample preparation and multiplex detection of panels of target nucleic acids in a sample comprising:
a. a sample preparation zone;
b. a multiplicity of three dimensional capture probe platforms for capturing specific classes of target molecules, said three dimensional platforms further comprising sets of platforms, said sets further being physically separated from one another, said sets further having capture probes that are specific for capturing any one specific class of said target molecule; and
c. spacer elements for separating said sets of three dimensional capture probe platforms, said sets and spacer elements further arranged so as to allow a sample to pass in succession from one set through a spacer element to another set.

51. An apparatus according to claim 50 wherein said capture probe platforms are transparent to visible light transmission.

52. An apparatus according to claim 50 wherein said spacer elements are opaque to visible light transmission.

53. An apparatus according to claim 50 wherein said spacer elements have an outer surface that has a high reflective index.

54. An apparatus according to claim 50 wherein said capture probe platforms have attached thereto a plurality of oligonucleotides.

55. An apparatus according to claim 54 wherein said oligonucleotides have a base sequence that is unique for each of said sets of platforms.

56. An apparatus according to claim 50 wherein said panel is directed to the multiplex detection of nucleic acids derived from an infective agent selected from the group consisting of a pathogenic microorganism, a bacterium, a protozoan, a virus, a fungus, and a prion.

57. An apparatus according to claim 50 wherein said capture probes of any given set of said sets are complementary to nucleic acid probes that are themselves complementary in part to nucleic acids derived from a source selected from the group consisting of a microorganism, a bacterium, a protozoan, a virus, and a fungus.

58. An apparatus according to claim 50 further comprising the elements of:
a. a inert filter zone for filtering particulate material from a sample;
b. a porous element zone for filtering small ionic materials from a sample;
c. a multiplicity of zones comprising enzymes for carrying out digestions of sample-derived materials;
d. a sample temperature adjustment zone; and
e. a multiplicity of re-circulation zones wherein a sample may be imported into said apparatus, said elements of (a), (b), (c), (d), and (e) further being connected together in any sequence while a sample may pass from one element to another and thereafter may further pass to said sets.

59. An apparatus for carrying out sample preparation and multiplex detection of panels of antigens in a sample comprising:
a. a sample preparation zone;
b. a multiplicity of three dimensional capture probe platforms for capturing specific classes of target molecules, said three dimensional platforms further comprising sets of platforms, said sets further being physically separated from one another, said sets further having capture probes that are specific for capturing any one specific class of said target molecule; and
c. spacer elements for separating said sets of three dimensional capture probe platforms, said sets and spacer elements further arranged so as to allow a sample to pass in succession from one set through a spacer element to another set.

60. An apparatus according to claim 59 wherein said capture probe platforms are transparent to visible light transmission.

61. An apparatus according to claim 59 wherein said spacer elements are opaque to visible light transmission.

62. An apparatus according to claim 59 wherein said spacer elements have an outer surface that has a high reflective index.

63. An apparatus according to claim 59 wherein said capture probe platforms have attached thereto a plurality of oligonucleotides.

64. An apparatus according to claim 63 wherein said oligonucleotides have a base sequence that is unique for each of said sets of platforms.

65. An apparatus according to claim 59 wherein said panel is directed to the multiplex detection of antigens derived from an infective agent selected from the group consisting of a pathogenic microorganism, a bacterium, a protozoan, a virus, a fungus, and a prion.

66. An apparatus according to claim 59 wherein said capture probes of any given set of said sets are complementary to nucleic acid sequences that are themselves attached to antibodies specific for antigens derived from a source selected from the group consisting of a drug metabolite, a microorganism, a bacterium, a protozoan, a virus, and a fungus.

67. An apparatus according to claim 59 further comprising the elements of:
a. a inert filter zone for filtering particulate material from a sample;
b. a porous element zone for filtering small ionic materials from a sample;
c. a multiplicity of zones comprising enzymes for carrying out digestions of sample-derived materials;
d. a sample temperature adjustment zone; and
e. a multiplicity of re-circulation zones wherein a sample may be imported into said apparatus, said elements of (a), (b), (c), (d), and (e) further being connected together in any sequence while a sample may pass from one element to another and thereafter may further pass to said sets.

68. A method of carrying out multiplex detection of target nucleic acids in a sample comprising the steps of:
a. providing a sample containing target nucleic acids of interest;
b. treating said sample with a sample buffer to form a pre-processed sample;
c. passing said pre-processed sample over an apparatus of claim 58;
d. capturing said target nucleic acids by capture probes of said apparatus of claim 58;
e. reacting a label with a signal probe, said signal probe having specificity for at least one other signal probe that is specific for said target of (d); and
f. detecting said reacted label in any of said sets of said apparatus of claim 58.

69. A method of carrying out multiplex detection of antigens in a sample comprising the steps of:
a. providing a sample containing target antigens of interest;
b. treating said sample with a sample buffer to form a pre-processed sample;
c. passing said pre-processed sample over an apparatus of claim 67;
d. capturing said target antigens by capture probes of said apparatus of claim 67;
e. reacting a label with a signal probe, said signal probe having specificity for at least one other signal probe that is specific for said target of (d); and
f. detecting said reacted label in any of said sets of said apparatus of claim 67.

70. A method according to claim 68 for detecting a panel of pathogenic agents selected from the group consisting of Salmonella sp., enterotoxigenic E. coli strains, Shigella sp., Campylobacter sp., Listeria sp., Yersinia sp., Vibrio cholerae, N. gonorrhea, Trichomonas, Chlamydia, Candida, Giardia lamblia, Cryptosporidium, and total coliforms.

71. A method according to claim 69 for detecting a panel of pathogenic agents selected from the group consisting of Salmonella sp., enterotoxigenic E. coli strains, Shigella sp., Campylobacter sp., Listeria sp., Yersinia sp., Vibrio cholerae, N. gonorrhea, Trichomonas, Chlamydia, Candida, Giardia lamblia, Cryptosporidium, and total coliforms.

72. A method according to claim 68 for detecting a panel of disease states selected from the group consisting of acquired immune deficiency, hepatitis, venereal disease, respiratory disease, diarrheal illness, a viral infection, a bacterial infection, a protozoan infection, heart disease, and cancer.

73. A method according to claim 69 for detecting a panel of disease states selected from the group consisting of acquired immune deficiency, hepatitis, venereal disease, respiratory disease, diarrheal illness, a viral infection, a bacterial infection, a protozoan infection, heart disease, and cancer.

74. A method according to claim 68 wherein said panel is directed to the multiplex detection of a viral agent selected from the group consisting of RSV, HPV, HSV, HIV, HCV, HBV, HTLV-1, HTLV-2, HGV, hepatitis D, and hepatitis E.

75. A method according to claim 69 wherein said panel is directed to the multiplex detection of a viral agent selected from the group consisting of RSV, HPV, HSV, HIV, HCV, HBV, HTLV-1, HTLV-2, HGV, hepatitis D, and hepatitis E.

76. A method according to claim 68 wherein said panel is directed to the multiplex detection of hepatitis B associated targets selected from the group consisting of HBV viral load, HBV genotype or serotype, ALT, AST, drug resistance, and drug monitoring.

77. A method according to claim 69 wherein said panel is directed to the multiplex detection of hepatitis B associated targets selected from the group consisting of HBV viral load, HBV genotype or serotype, ALT, AST, drug resistance, and drug monitoring.

78. A method according to claim 68 wherein said panel is directed to the multiplex detection of hepatitis C associated targets selected from the group consisting of HCV viral load, HCV genotype or serotype, ALT, AST, drug resistance, and drug monitoring.

79. A method according to claim 69 wherein said panel is directed to the multiplex detection of hepatitis C associated targets selected from the group consisting of HCV viral load, HCV genotype or serotype, ALT, AST, drug resistance, and drug monitoring.

80. A method according to claim 68 wherein said panel is directed to the multiplex detection of a microorganism selected from the group consisting of Salmonella sp., enterotoxigenic E. coli strains, Shigella sp., Campylobacter sp., Listeria sp., Yersinia sp., and Vibrio cholerae.

81. A method according to claim 69 wherein said panel is directed to the multiplex detection of a microorganism selected from the group consisting of Salmonella sp., enterotoxigenic E. coli strains, Shigella sp., Campylobacter sp., Listeria sp., Yersinia sp., and Vibrio cholerae.

82. A method according to claim 68 wherein said panel is directed to the multiplex detection of a of a virus or other target molecule associated with hepatitis infection and treatment selected from the group consisting of hepatitis A, hepatitis B, hepatitis C, hepatitis D, hepatitis E, hepatitis G, ALT, AST, and hepatitis drug metabolites.

83. A method according to claim 69 wherein said panel is directed to the multiplex detection of a of a virus or other target molecule associated with hepatitis infection and treatment selected from the group consisting of hepatitis A, hepatitis B, hepatitis C, hepatitis D, hepatitis E, hepatitis G, ALT, AST, and hepatitis drug metabolites.

84. A method according to claim 68 wherein said panel is directed to the multiplex detection of a pathogenic agent selected from the group consisting of acquired immune deficiency virus, N. gonorrhea, Trichomonas, T. pallidum, Chlamydia, Candida, HPV, and HSV.

85. A method according to claim 69 wherein said panel is directed to the multiplex detection of a pathogenic agent selected from the group consisting of acquired immune deficiency virus, N. gonorrhea, Trichomonas, T. pallidum, Chiamydia, Candida, HPV, and HSV.

86. A method according to claim 68 wherein said panel is directed to the multiplex detection of a microorganism selected from the group consisting of Giardia lamblia, Cryptosporidium parvum, Entamoeba histolytica, and total coliforms.

87. A method according to claim 69 wherein said panel is directed to the multiplex detection of a microorganism selected from the group consisting of Giardia lamblia, Cryptosporidium parvum, Entamoeba histolytica, and total coliforms.

88. A method according to claim 68 wherein said panel is directed to the multiplex detection of nucleic acids derived from a source selected from the group consisting of a microorganism, a bacterium, a protozoan, a virus, and a fungus.

89. A method according to claim 69 wherein said panel is directed to the multiplex detection of antigens derived from a source selected from the group consisting of a microorganism, a bacterium, a protozoan, a virus, and a fungus.

90. A method according to claim 68 wherein said panel is directed to the multiplex detection of respiratory pathogens and/or resistance of such pathogens to anti-TB drugs selected from the group consisting of a pneumonia causing bacteria, RSV, M. africanum, M. bovis, M. microti, and M. tuberculosis.

91. A method according to claim 69 wherein said panel is directed to the multiplex detection of respiratory pathogens and/or resistance of such pathogens to anti-TB drugs selected from the group consisting of a pneumonia causing bacteria, RSV, M. africanum, M. bovis, M. microti, and M. tuberculosis.

92. An apparatus according to claim 50 that is a track-based instrument to automate assays.

93. A method according to claim 68 wherein the apparatus of claim 58 is a track-based instrument to automate assays.

94. A method according to claim 69 wherein the apparatus of claim 67 is a track-based instrument to automate assays.


SEQUENCE LISTING (79 sequences)

SEQ ID NO: 1 - DNA, 12 bp, SYNTHETIC
ggcactacacgg

SEQ ID NO: 2 - DNA, 12 bp, SYNTHETIC
atcggtgacgct

SEQ ID NO: 3 - DNA, 12 bp, SYNTHETIC
gacggcgatgta

SEQ ID NO: 4 - DNA, 12 bp, SYNTHETIC
atgacggttgac

SEQ ID NO: 5 - DNA, 12 bp, SYNTHETIC
acgcaatgtggt

SEQ ID NO: 6 - DNA, 12 bp, SYNTHETIC
acgacactggtt

SEQ ID NO: 7 - DNA, 12 bp, SYNTHETIC
cgatgccgacaa

SEQ ID NO: 8 - DNA, 12 bp, SYNTHETIC
ccgcagcattgg

SEQ ID NO: 9 - DNA, 12 bp, SYNTHETIC
ttggcacgcact

SEQ ID NO: 10 - DNA, 12 bp, SYNTHETIC
ttgccacgccga

SEQ ID NO: 11 - DNA, 12 bp, SYNTHETIC
cggctatggcaa

SEQ ID NO: 12 - DNA, 12 bp, SYNTHETIC
cgactgatgtgc

SEQ ID NO: 13 - DNA, 12 bp, SYNTHETIC
tacgatggcagt

SEQ ID NO: 14 - DNA, 12 bp, SYNTHETIC
ccgtggtcaaca

SEQ ID NO: 15 - DNA, 12 bp, SYNTHETIC
ctatgcgacggt

SEQ ID NO: 16 - DNA, 12 bp, SYNTHETIC
tcggctgtcgca

SEQ ID NO: 17 - DNA, 12 bp, SYNTHETIC
taacggtgtcgg

SEQ ID NO: 18 - DNA, 12 bp, SYNTHETIC
cacgataacggc

SEQ ID NO: 19 - DNA, 12 bp, SYNTHETIC
ccattgcggctg

SEQ ID NO: 20 - DNA, 12 bp, SYNTHETIC
caacagcatcgg

SEQ ID NO: 21 - DNA, 12 bp, SYNTHETIC
ccgtgtagtgcc

SEQ ID NO: 22 - DNA, 12 bp, SYNTHETIC
agcgtcaccgat

SEQ ID NO: 23 - DNA, 12 bp, SYNTHETIC
tacatcgccgtc

SEQ ID NO: 24 - DNA, 12 bp, SYNTHETIC
gtcaaccgtcat

SEQ ID NO: 25 - DNA, 12 bp, SYNTHETIC
accacattgcgt

SEQ ID NO: 26 - DNA, 12 bp, SYNTHETIC
aaccagtgtcgt

SEQ ID NO: 27 - DNA, 12 bp, SYNTHETIC
ttgtcggcatcg

SEQ ID NO: 28 - DNA, 12 bp, SYNTHETIC
ccaatgctgcgg

SEQ ID NO: 29 - DNA, 12 bp, SYNTHETIC
agtgcgtgccaa

SEQ ID NO: 30 - DNA, 12 bp, SYNTHETIC
tcggcgtggcaa

SEQ ID NO: 31 - DNA, 12 bp, SYNTHETIC
ttgccatagccg

SEQ ID NO: 32 - DNA, 12 bp, SYNTHETIC
gcacatcagtcg

SEQ ID NO: 33 - DNA, 12 bp, SYNTHETIC
actgccatcgta

SEQ ID NO: 34 - DNA, 12 bp, SYNTHETIC
tgttgaccacgg

SEQ ID NO: 35 - DNA, 12 bp, SYNTHETIC
accgtcgcatag

SEQ ID NO: 36 - DNA, 12 bp, SYNTHETIC
tgcgacagccga

SEQ ID NO: 37 - DNA, 12 bp, SYNTHETIC
ccgacaccgtta

SEQ ID NO: 38 - DNA, 12 bp, SYNTHETIC
gccgttatcgtg

SEQ ID NO: 39 - DNA, 12 bp, SYNTHETIC
cagccgcaatgg

SEQ ID NO: 40 - DNA, 12 bp, SYNTHETIC
ccgatgctgttg

SEQ ID NO: 41 - DNA, 36 bp, SYNTHETIC
  bases 12 and 13 linked with hydrophilic spacer
ccgtgtagtgccccgtgtagtgccccgtgtagtgcc

SEQ ID NO: 42 - DNA, 36 bp, SYNTHETIC; identical to SEQ ID NO: 41

SEQ ID NO: 43 - DNA, 12 bp, SYNTHETIC
tgtttagccctg

SEQ ID NO: 44 - DNA, 12 bp, SYNTHETIC
caatgctgctcg

SEQ ID NO: 45 - DNA, 12 bp, SYNTHETIC
ttctgggtccta

SEQ ID NO: 46 - DNA, 12 bp, SYNTHETIC
cccattcaagta

SEQ ID NO: 47 - DNA, 12 bp, SYNTHETIC
agaactccacag

SEQ ID NO: 48 - DNA, 12 bp, SYNTHETIC
ctttaccttgct

SEQ ID NO: 49 - DNA, 12 bp, SYNTHETIC
agcaagccaact

SEQ ID NO: 50 - DNA, 12 bp, SYNTHETIC
cagggctaaaca

SEQ ID NO: 51 - DNA, 12 bp, SYNTHETIC
cgagcagcattg

SEQ ID NO: 52 - DNA, 12 bp, SYNTHETIC
taggacccagaa

SEQ ID NO: 53 - DNA, 12 bp, SYNTHETIC
tacttgaatggg

SEQ ID NO: 54 - DNA, 12 bp, SYNTHETIC
ctgtggagttct

SEQ ID NO: 55 - DNA, 12 bp, SYNTHETIC
agcaaggtaaag

SEQ ID NO: 56 - DNA, 12 bp, SYNTHETIC
agttggcttgct

SEQ ID NO: 57 - DNA, 15 bp, HCV
tgcacggtctacgag

SEQ ID NO: 58 - DNA, 18 bp, HCV
ctcgcaagcaccctatca

SEQ ID NO: 59 - DNA, 15 bp, HCV
ggcagtaccacaagg

SEQ ID NO: 60 - DNA, 14 bp, HCV
ccggttccgcagac

SEQ ID NO: 61 - DNA, 15 bp, HCV
ctatggctcttccgg

SEQ ID NO: 62 - DNA, 14 bp, HCV
tactaacgccatgg

SEQ ID NO: 63 - DNA, 15 bp, HCV
gcgtgaagacagtag

SEQ ID NO: 64 - DNA, 27 bp, COMBINATION SYNTHETIC AND HCV
  bases 15 and 16 linked with hydrophilic spacer
tgcacggtctacgagatcggtgacgct

SEQ ID NO: 65 - DNA, 30 bp, COMBINATION SYNTHETIC AND HCV
  bases 18 and 19 linked with hydrophilic spacer
ctcgcaagcaccctatcaatcggtgacgct

SEQ ID NO: 66 - DNA, 27 bp, COMBINATION SYNTHETIC AND HCV
  bases 15 and 16 linked with hydrophilic spacer
ggcagtaccacaaggatcggtgacgct

SEQ ID NO: 67 - DNA, 26 bp, COMBINATION SYNTHETIC AND HCV
  bases 14 and 15 linked with hydrophilic spacer
ccggttccgcagacatcggtgacgct

SEQ ID NO: 68 - DNA, 27 bp, COMBINATION SYNTHETIC AND HCV
  bases 15 and 16 linked with hydrophilic spacer
ctatggctcttccggatcggtgacgct

SEQ ID NO: 69 - DNA, 26 bp, COMBINATION SYNTHETIC AND HCV
  bases 14 and 15 linked with hydrophilic spacer
tactaacgccatggatcggtgacgct

SEQ ID NO: 70 - DNA, 27 bp, COMBINATION SYNTHETIC AND HCV
  bases 15 and 16 linked with hydrophilic spacer
gcgtgaagacagtagatcggtgacgct

SEQ ID NO: 71 - DNA, 27 bp, COMBINATION SYNTHETIC AND HCV
  bases 15 and 16 linked with hydrophilic spacer
tgcacggtctacgagcagggctaaaca

SEQ ID NO: 72 - DNA, 30 bp, COMBINATION SYNTHETIC AND HCV
  bases 18 and 19 linked with hydrophilic spacer
ctcgcaagcaccctatcacagggctaaaca

SEQ ID NO: 73 - DNA, 27 bp, COMBINATION SYNTHETIC AND HCV
  bases 15 and 16 linked with hydrophilic spacer
ggcagtaccacaaggcagggctaaaca

SEQ ID NO: 74 - DNA, 26 bp, COMBINATION SYNTHETIC AND HCV
  bases 14 and 15 linked with hydrophilic spacer
ccggttccgcagaccagggctaaaca

SEQ ID NO: 75 - DNA, 27 bp, COMBINATION SYNTHETIC AND HCV
  bases 15 and 16 linked with hydrophilic spacer
ctatggctcttccggcagggctaaaca

SEQ ID NO: 76 - DNA, 26 bp, COMBINATION SYNTHETIC AND HCV
  bases 14 and 15 linked with hydrophilic spacer
tactaacgccatggcagggctaaaca

SEQ ID NO: 77 - DNA, 27 bp, COMBINATION SYNTHETIC AND HCV
  bases 15 and 16 linked with hydrophilic spacer
gcgtgaagacagtagcagggctaaaca

SEQ ID NO: 78 - DNA, 27 bp, COMBINATION SYNTHETIC AND HCV
  bases 15 and 16 linked with hydrophilic spacer
ggcagtaccacaaggatcggtgacgct

SEQ ID NO: 79 - DNA, 26 bp, COMBINATION SYNTHETIC AND HCV
  bases 14 and 15 linked with hydrophilic spacer
tactaacgccatggatcggtgacgct